(19) United States
(12) Patent Application Publication
(10) Pub. No.: US 2005/0187161 A1
(43) Pub. Date: Aug. 25, 2005

(54) Biopanning as an approach to study the pathogenesis of and produce novel treatment modalities for invasive Aspergillosis

(75) Inventors: Dimitrios P. Kontoyiannis, Bellaire, TX (US); Wadih Arap, Houston, TX (US); Renata Pasqualini, Houston, TX (US); Michail S. Lionakis, Houston, TX (US); Johanna Lahdenranta, Houston, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/940,004

(22) Filed: Sep. 13, 2004

Related U.S. Application Data
(60) Provisional application No. 60/502,509, filed on Sep. 12, 2003.

(51) Int. Cl. A61K 38/08; A61K 38/10

(52) U.S. Cl. 514/14; 514/16; 514/17; 514/15

(57) ABSTRACT

The present invention concerns novel methods of identifying peptide sequences that selectively bind to fungal surface molecules. The general method, Biopanning and Rapid Analysis of Selective Interactive Ligands (BRASIL) provides for rapid and efficient separation of phage that bind to fungal surface molecules. BRASIL may be used in a pre-selection procedure to subtract phage that bind non-specifically to a first target, before exposing the subtracted library to a second target. Certain embodiments concern peptides identified by BRASIL against fungal surface components and methods of use of such peptides for delivery of therapeutic agents or imaging agents or diagnosis or treatment of fungal pathogenesis such as Invasive Aspergillus (IA). Novel compositions and treatments for IA as well as other fungal infection in immunocompromised patients are also disclosed.

This application claims priority to U.S. Provisional Patent application Ser. No. 60/502,509, filed on Sep. 12, 2003, entitled “Biopanning as an approach to study the pathogenesis of and produce novel treatment modalities for invasive Aspergillosis,” which is incorporated herein by reference in its entirety.

### BACKGROUND OF THE INVENTION

I. Field of the Invention

The present invention concerns the fields of molecular and cellular biology, immunobiology, molecular medicine, and molecular diagnostics. Specifically, the present invention relates to diagnostic and therapeutic compositions and methods comprising a ligand that binds to a fungal cell, for example an Aspergillus cell.

II. Description of Related Art

Fungal infections in humans can range from common, mild superficial infections such as athlete's foot and vaginal and oral thrush to serious life-threatening diseases such as invasive aspergillosis. The yeasts responsible for thrush form part of the normal commensal flora in humans, living harmlessly on skin, respiratory, gastrointestinal and genital tracts until a change in the host allows them to cause infection. The dermatophyte fungi, which cause athlete's foot and other infections of the skin, hair and nails are dependent on a human or animal host and are passed from person to person or animal to person. Most fungi, however are free living in the environment and few of these are capable of causing infection in an otherwise healthy individual but can be responsible for life-threatening infections in patients with lowered immunity.

Invasive aspergillosis (IA) has emerged as one of the common causes of infection in patients with hematological malignancies (e.g., acute leukemia) and bone marrow transplantation (BMT). In particular, Aspergillus fumigatus is one of the most common fungal species causing IA. Lung infection is one of the predominant types of IA. Fungal infections in immunocompromised patients may also include infections of non-Aspergillus filamentous fungi, such as various hyalohyphomycetes (e.g., Fusarium species, Scedosporium species), phaeohyphomycetes (e.g., Exophila species) and/or Zygomycetes. IA has a poor outcome with reported mortality rates approaching 80% in some immunosupressed patient populations (Kontoyiannis and Bodey, 2002). The reason of the sub-optimal efficacy of current strategies is due mainly to the fact that the disease is diagnosed late when the fungal burden in the lungs is substantial. In that setting current antifungal therapies have a mediocre effect at best, especially in profoundly compromised hosts.

In immunocompetent hosts, there are two lines of defense against inhaled Aspergillus fumigatus conidia (filamentous fungi typically have two developmental stages of growth, the conidia stage and the hyphae stage). 1) The resident lung macrophages are responsible for phagocytosis and nonoxidative killing of conidia (Clemons et al., 2000). These cells have a very efficient phagocytic capacity, with more than 10⁸ conidia phagocytosed per day (Latge, 1999). Still, some conidia ultimately escape phagocytosis, germinate, and establish an invasive infection. 2) Once an infection is established, neutrophils are chemotactically attracted to and attach to the hyphae. Hyphal elements are subsequently destroyed extracellularly by the oxidative cytotoxic mechanisms of polymorphonuclear leukocytes (PMNs) (Latge, 1999, Clemons et al., 2000). Nevertheless, in patients with inherited biological defects (e.g., patients with chronic granulomatous disease) or iatrogenic (treatment induced) conditions (e.g., cancer patients, transplant recipients, patients with chronic inflammatory disorders) these lines of defense are defective, rendering a high risk for the acquisition of IA (Balow et al., 1975). Finally inhalation of Aspergillus conidia is implicated in allergic manifestations in predisposed, otherwise immunocompetent persons, e.g., allergic bronchopulmonary aspergillosis (ABPA), and allergic Aspergillus sinusitis.

Additional compositions and methods are needed for the treatment or diagnosis of fungal infections in subjects that are susceptible to these types of infections.

### SUMMARY OF THE INVENTION

Systemic fungal infection is becoming more and more common in modern hospitals, e.g., candidiasis and aspergillosis, as well as histoplasmosis, blastomycosis, and coccidioidomycosis. Severe systemic fungal infection in hospitals is commonly seen in three major settings: in patients following chemotherapy, and other oncology patients with immune suppression; immune compromised due to Acquired Immune Deficiency Syndrome caused by HIV infection; and patients in intensive care (ICU) that are compromised due to the presence of long-term intravascular lines, severe systemic illness, burns, and/or prolonged antibiotic therapy. Thus, additional compositions and methods are needed for the treatment and diagnosis of fungal infections.

Embodiments of the invention include compositions comprising an isolated peptide having at least 3, 4, 5, 6, 7, or more contiguous amino acids of a sequence selected using methods described herein. In certain aspects an isolated peptide sequence may be selected from the group consisting of SEQ ID NO: 1 through SEQ ID NO:16, wherein said peptide binds to fungal cells. The peptide may be 5, 6, 7, 8, 9, 10, 11, 12, 13, 14, 15, 16, 17, 18, 19, 20, 25, 30, 35, 40, 45, 50, 60, 70, 80, 90, or a 100 or fewer amino acids is length, or lengths therebetween. In preferred aspects the isolated peptide is 50 amino acids or less in size. Further preferred is an isolated peptide of 25 amino acids or less in size. Still further preferred is an isolated peptide of 10 amino acids or less in size. Yet still further preferred is an isolated peptide of 9 amino acids or less in size. Even more preferred is an isolated peptide of 7 amino acids or less in size. In certain aspects an isolated peptide comprises at least 5 contiguous amino acids of a sequence selected from the group consisting of SEQ ID NO: 1 through SEQ ID NO:16. In other aspects the peptide is at most 100 residues in length and may be in the range of 3, 4, 5, 6, 7, 8, 9, 10, 15, 20 to 10, 11, 12, 13, 14, 15, 20, 25, 30, 35, 40, 45 or 50 residues. The peptide may be a cyclic peptide. In preferred embodiments the peptide may include cysteine residues at either or both peptide terminus.

The peptide may be operatively coupled to an agent to be delivered to a fungal cell, preferably covalently coupled to the agent. The agent may be a drug, a chemotherapeutic agent, a radioisotope, an anti-fungal agent, a peptide, a protein, an antibiotic, an antibody, a Fab fragment of an antibody, an imaging agent, a cell, a vector or a virus. In certain aspects of the invention the fungal cell is an Aspergillus, Fusarium, Zygomycetes or Scedosporium cell. An anti-fungal agent may comprise one or more agents including voriconazole, posaconazole, clotrimazole, miconazole, ketoconazole, econazole, butoconazole, oxiconazole, terconazole, itraconazole, ravuconazole, fluconazole, amphotericin B, amphotericin B lipid formulation, liposomal amphotericin B, ABLC, nystatin, nystatin lipid formulation, an azole, terbinafine, echinocandin, terbinafine, naftifine, tolnaftate, mediocidin, candicidin, pimaricin, trichomycin, hamycin, aurefungin, ascosin, ayfattin, azacolutin, trichomycin, levorin, heptamycin, candimycin, perimycin, caspofungin, micafungin, anidulfungin or other known anti-fungal agents. The agent may be comprised in a liposome. In certain aspects a peptide of the invention is attached to a delivery vehicle, such as a liposome or may be attached to a support, such a solid support. Typically, peptides of the invention selectively bind to fungal cells. Compositions of the invention may further comprise lipids. In certain aspects the lipids include phospholipids in the form of liposomes.

Further embodiments of the invention include methods of selecting a fungal cell targeting peptide that include a) obtaining at least one sample comprising fungal cells; b) exposing the sample to a peptide library; and c) recovering one or more peptides that bind to the fungal cells. Typically the peptide library is a phage display library. In certain aspects phage are recovered by infecting pilus positive bacteria. The phage may be recovered by a) amplifying phage inserts using a variety nucleic acid amplification techniques known in the art; b) ligating the amplified inserts to phage DNA; and c) producing phage from the ligated DNA. In other aspects, phage may be recovered by using BRASIL (Biopanning and Rapid Analysis of Selective Interactive Ligands). The method may further comprising obtaining one or more types of non-fungal cells and exposing the cells to the peptide library and recovering one or more peptides that do not bind to the one or more types of non-fungal cells. The methods may further comprise a) preselecting the phage library against non-fungal cell type; b) removing phage that bind to the non-fungal cell type; and c) selecting the remaining phage against fungal cells of interest. In other aspects of the invention a conidia may be selected against hypae and vice versa to identify conida and/or hyphae targeting ligands. The fungal cells include, but are not limited to Aspergillus, Fusarium, Zygomycetes or Scedosporium cells.

Still further embodiments of the invention include methods of treating or ameliorating a fungal infection in a subject that comprise a) obtaining a fungal cell targeting peptide as described herein or as made by the methods described herein, wherein the peptide i) is operatively coupled to and delivers a therapeutic agent to the fungal cell, ii) inhibits the adhesion of the fungal cell to a tissue or organ, or inhibits the growth or kills the fungal cell, or iii) is operatively coupled to and delivers a therapeutic agent to the fungal cell and/or inhibits the adhesion of the fungal cell to a tissue or organ; and b) administering the peptide to the subject. In certain aspects the therapeutic agent slows, inhibits or otherwise has a negative effect on the viability of the fungal cell, or kills the fungal cell. The targeting peptide may comprise a peptide having at least 3, 4, 5, 6, 7, 8, 9, 10 or more contiguous amino acids of a sequence selected from the group consisting of SEQ ID NO: 1 through SEQ ID NO:16 or other sequence identified by the methods described herein, wherein said peptide binds to fungal cells. Aspects of the invention include a therapeutic agent that is a drug, a chemotherapeutic agent, a radioisotope, an anti-fungal agent, a peptide, a protein, an antibiotic, an antibody, a Fab fragment of an antibody, an imaging agent, a cell, a vector or a virus. An anti-fungal agent may comprise one or more agents including but not limited to voriconazole, posaconazole, clotrimazole, miconazole, ketoconazole, econazole, butoconazole, oxiconazole, terconazole, itraconazole, ravuconazole, fluconazole, amphotericin B, amphotericin B lipid formulation, liposomal amphotericin B, ABLC, nystatin, nystatin lipid formulation, an azole, terbinafine, echinocandin, terbinafine, naftifine, tolnaftate, mediocidin, candicidin, pimaricin, trichomycin, hamycin, aurefungin, ascosin, ayfattin, azacolutin, trichomycin, levorin, heptamycin, candimycin, perimycin, caspofungin, micafungin, anidulfungin or other known antifungal agents (see Manual of Clinical Microbiology,1999; The Use of Antibiotics: A Clinical Review of Antibacterial, Antifungal and Antiviral Drugs, 1997).

Embodiments of the invention include methods of targeting the delivery of an agent to a fungal cell in a subject. The methods may comprise a) obtaining a peptide composition as described herein or as made by methods described herein; b) operatively coupling the peptide to the agent; and c) administering the peptide-coupled agent to the subject. A subject is typically a human, but includes, and is not limited to a mouse, a dog, a cat, a rat, a sheep, a horse, a cow, a goat, a pig or other domestic or wild animals. Methods and compositions of the invention may also be used in environmental, industrial, and agricultural applications to control the growth of fungi. An agent may be a drug, a chemotherapeutic agent, a radioisotope, an anti-fungal agent, a peptide, a protein, an antibiotic, an antibody, a Fab fragment of an antibody, an antigen, an imaging agent, a cell, a vector or a virus. In certain embodiments, an isolated peptide of the invention may compete for fungal cell binding and thus be used as therapeutic by reducing the binding to an organ or tissue of a subject.

Further embodiments of the invention include methods of identifying a fungal cell, comprising a) contacting a sample or subject suspected of comprising a fungal cell with an isolated peptide as described herein or as made the methods described herein; and b) detecting binding of the peptide to the sample, thereby identifying sample as comprising fungal cells.

Still further embodiments of the invention include methods of identifying a receptor or protein that interacts with a fungal targeting peptide, comprising the steps of a) obtaining a composition suspected of comprising a receptor or protein that interacts with a fungal cell targeting peptide; b) contacting the composition with a peptide as described herein or as made by methods described herein under conditions that permit binding of the peptide to any such receptor or protein present in the composition; and c) identifying a receptor or protein that binds to the peptide. Typically the composition comprises fungal cells. The methods may further comprise isolating the receptor or protein, preparing an antibody or antibody fragment that recognizes and binds to the receptor or protein, and/or attaching an agent that one desires to have delivered to fungal cells to said antibody or antibody fragment. In other aspects of the invention one may use the targeting peptide to produce anti-idiotope antibodies that bind to the fungal protein that interacts or binds to a targeting peptide.

Yet still further embodiments of the invention include an antibody or antibody fragment that recognizes and binds to a receptor or protein identified by the methods of the invention. The antibody or antibody fragment may further comprise an agent or macromolecular complex that one desires to have delivered to fungal cells attached to the antibody or antibody fragment.

Embodiments of the invention also include methods of selectively targeting a fungal cell in a patient, comprising the steps of a) obtaining an antibody or antibody fragment in accordance with or prepared by methods described herein; and b) administering the antibody or fragment to the patient to thereby target the fungal cells.

As used herein, a “phage display library” means a collection of phage particles that has been genetically engineered to express a set of putative targeting peptides on their outer surface. In preferred embodiments, DNA sequences encoding the putative targeting peptides are inserted in frame into a gene encoding a phage capsule protein. In other preferred embodiments, the putative targeting peptide sequences are in part random mixtures of all twenty amino acids and in part non-random. In certain preferred embodiments the putative targeting peptides of the phage display library exhibit one or more cysteine residues at fixed locations within the targeting peptide sequence.

A “receptor” for a targeting peptide includes but is not limited to any molecule or macromolecular complex that binds to a targeting peptide. Non-limiting examples of receptors include peptides, proteins, glycoproteins, lipoproteins, epitopes, lipids, carbohydrates, multi-molecular structures, and a specific conformation of one or more molecules. In preferred embodiments, a “receptor” is a naturally occurring molecule or complex of molecules that is present on the surface of cells within a target organism, organ, tissue or cell type. More preferably, a “receptor” is a naturally occurring molecule or complex of molecules that is present on the surface of fungal cells.

A “subject” refers generally to a mammal. In certain embodiments, the subject is a horse, dog, cat, mouse, rabbit, or bird. In other embodiments, the subject is a human.

As used herein in the specification, “a” or “an” may mean one or more. As used herein in the claim(s), in conjunction with the word “comprising,” the words “a” or “an” may mean one or more than one. As used herein “another” may mean at least a second or more of an item.

Certain embodiments concern methods of obtaining antibodies against an antigen. In preferred embodiments, the antigen comprises one or more targeting peptides. The targeting peptides are prepared and immobilized on a solid support, serum-containing antibodies is added and antibodies that bind to the targeting peptides are collected.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Illustrates an exemplary method of the BRASIL method using a CX₇C Phage Library.

FIG. 2 Illustrates an example of a flow chart for applying the BRASIL method to identify ligands of A. fumigatus conidia or hyphae surface proteins.

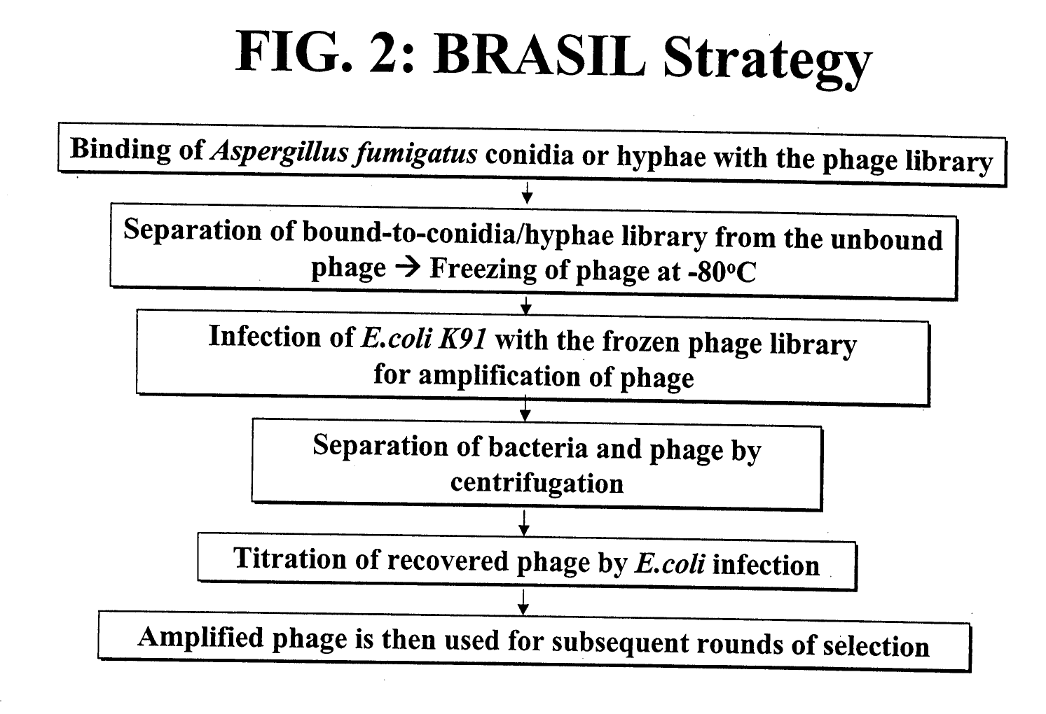

FIG. 3 Illustrates an example of a flow chart for conditions of incubating a Phage library with either A. fumigatus conidia or hyphae.

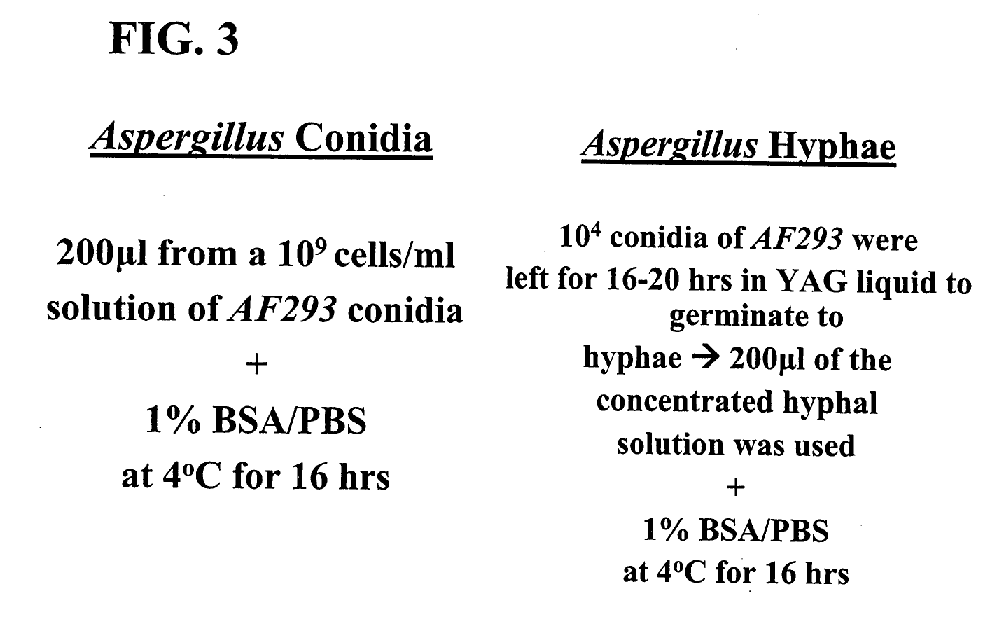

FIG. 4 Illustrates examples of peptide motifs identified after 4 selection rounds for A. fumigatus conidia.

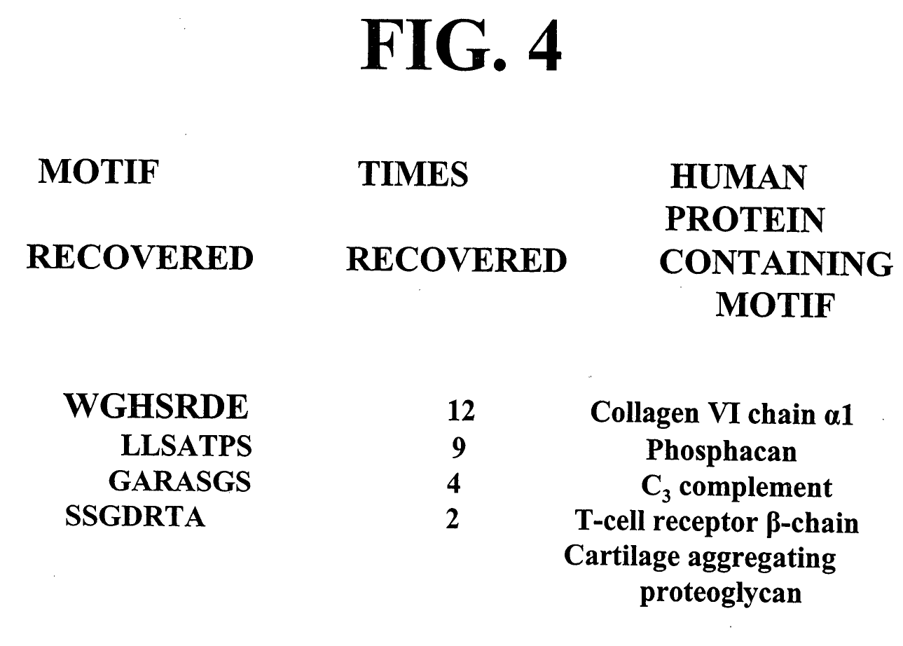

FIG. 5 Illustrates an example of the WGHSRDE (SEQ ID NO:1) Motif on the surface of collagen VI.

FIG. 6 Illustrates an example of the WGHSRDE (SEQ ID NO:1) Motif on the surface of collagen VI.

FIG. 7 Illustrates examples of peptide motifs identified after 5 selection rounds for A. fumigatus hyphae.

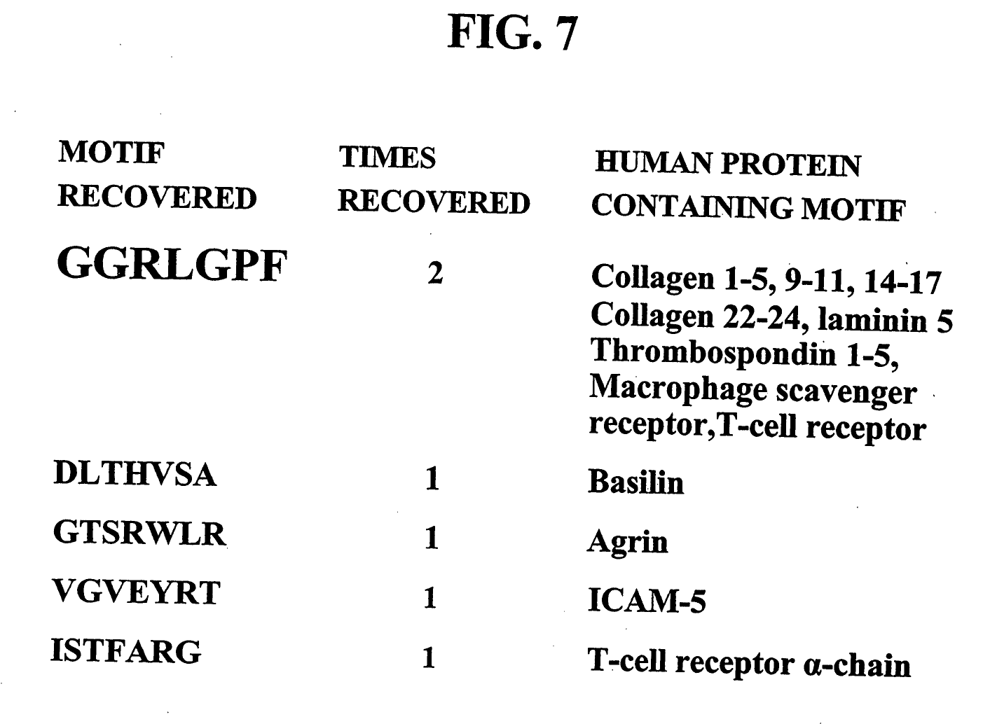

FIG. 8A-8C Illustrates an exemplary fungal cell wall and membrane (FIG. 8A). FIGS. 8B and 8C illustrate an exemplary location of the glucan synthase complex on the apical tips of Aspergillus hyphae.

FIG. 9 Illustrates an exemplary method of a DiBAC staining of dead CAS-treated Aspergillus fumigatus hyphae.

FIG. 10. BRASIL method for identification of potential host ligands that bind on the surface of Aspergillus fumigatus hyphae (upper) and conidia (lower).

FIG. 11 Illustrates an exemplary method of a murine model of acute invasive pulmonary aspergillosis.

FIG. 12 Illustrates an exemplary method of in vivo phage-display screening for peptides that home to mouse lung tissue in the presence of a lethal challenge of acute pulmonary invasive aspergillosis.

FIG. 13: illustrates specific inhibition of the WGHSRDE (SEQ ID NO:1) phage binding on AF293 conidia by the WGHSRDE (SEQ ID NO: 1) synthetic peptide.

FIG. 14 represents an example of WGHSRDE (SEQ ID NO: 1) Peptide Protection in Lethal Challenge of Acute Pulmonary Aspergillosis.

### DESCRIPTION OF ILLUSTRATIVE EMBODIMENTS

Systemic fungal infection is becoming more and more common in modern hospitals, e.g., candidiasis and aspergillosis, as well as histoplasmosis, blastomycosis, and coccidioidomycosis. Severe systemic fungal infection in hospitals is commonly seen in three major settings: in patients following chemotherapy, and other patients with immune suppression; immune compromised due to Acquired Immune Deficiency Syndrome caused by HIV infection; and patients in intensive care (ICU) and are compromised due to the presence of long-term intravascular lines, severe systemic illness, burns, and/or prolonged antibiotic therapy.

The exact pathogenesis of IA and the early molecular events underlying the attachment of conidia to host tissue receptors and the transition from conidia to an invasive infection by hyphae remain unclear. Current approaches to study Aspergillus pathogenesis have failed (Chaveroche et al., 2000) and more comprehensive strategies are needed. More specifically, the strategy of Biopanning and Rapid Analysis of Selective Interactive Ligands (BRASIL), is contemplated for the screening and identification of cell-surface-binding peptides from phage display libraries (Giordano et al., 2001, Arap et al., 2002, Koivunen et al., 1999). Peptides identified using these methods may be used to identify potential peptides and/or host ligands that bind on the surface of a fungal organism such as A. fumigatus conidia and hyphae. This approach will be of significant scientific and medical utility in the delineation of the pathogenesis of and/or the treatment of invasive mycoses in profoundly immunocompromised patients. A need exists in the art for an effective means of identifying peptides and/or host ligands that bind to and possibly prevent the deleterious effects of fungal infection, especially in immunocompromised subjects.

Additional therapeutic compositions and methods for the treatment and therapy of fungal infections are still needed, in particular methods and compositions related to Aspergillosis. In one aspect of the invention, the inventors have employed Biopanning and Rapid Analysis of Selective Interactive Ligands (BRASIL) to identify peptides that selectively bind to a variety of fungal cells. As used herein “selective binding” in no way precludes binding to other cells or material, but connotes the preferential binding of fungal cells. Selective binding may include a 2, 3, 4, 5, 6, 7, 8, 9, 10 or more fold preference for fungal cells as compared to non-fungal cells. Methods and compositions for identification and use of targeted peptides (host ligands) that bind to the surface of invasive organisms (e.g., fungi) are disclosed.

In one example, fungal cells were profiled, including members of the genus Aspergillus. Screening the cells with a CX₇C random phage library, for example, yielded several peptide motifs that bound fungal cells (SEQ ID NO: 1 to SEQ ID NO: 16) exhibited high frequency binding as compared to the control insert-less phage. Comparison of the selected motifs with available sequences in on-line protein databases suggests that a number of proteins share homologous sequences with these peptides. These peptides are being use in further studies to identify and purify protein(s) that interact, directly or indirectly, with an identified peptide, including identifying and purifying corresponding receptor(s). In the clinics the newly identified peptides and peptide motifs may serve as targeting moieties, drugs and/or drug leads. Also, the identified peptides can be optimized as delivery vehicles or enhancers for targeted therapy of fungal infections.

TABLE 1

Motifs identified after 4 selection rounds for
conidia and 5 selection rounds for hyphae.

| | | |
|---|---|---|
| Aspergillus target | Times | |
| peptide and motif | identified | Human protein containing motif (Protein description) |

| | | |
|---|---|---|
| Conidia | | |
| WGHSRDE | 12/40 | Procollagen VI, chain alpha 1 |
| (SEQ ID NO:1) | | (Collagen component) |
| LLSATPS | 8/40 | Phosphacan |
| (SEQ ID NO:2) | | (Proteoglycan component) |
| GARASGS | 4/40 | C3 molecule |
| (SEQ ID NO:3) | | (Complement component) |
| ELLRRGGS | 2/40 | Lymphokine alpha-kinase |
| (SEQ ID NO:4) | | (Membrane receptor) |
| SSGDRTA | 2/40 | Cartilage aggregating proteoglycan T-cell receptor beta-chain |
| (SEQ ID NO:5) | | (Proteoglycan component) |
| SLVRGGT | 2/40 | C9 molecule |
| (SEQ ID NO:6) | | (Complement component) |
| IGRAPQM | 2/40 | MHC I heavy chain |
| (SEQ ID NO:7) | | (Major histocompatibility complex) |
| GGWGPGS | 2/40 | Properdin precursor (complement molecule), thrombospondin-1 |
| (SEQ ID NO:8) | | (molecule of the extracellular matrix), chemokine ligand |
| | | (membrane receptor) |
| Hyphae | | |
| GGRLGPF | 2/40 | Procollagen 1-7, 9-11, 14-17, 19, 22, 24 (collagen components) |
| (SEQ ID NO:9) | | thrombospondin 1-5 (molecules of the extracellular matrix), |
| | | laminin 5 macrophage scavenger receptor (cellular immune |
| | | response) T-cell receptor beta-chain (cellular immune response) |
| DLTHVSA | 1/40 | Basilin |
| (SEQ ID NO:10) | (elastin-like molecule) | |
| GTSRWLR | 1/40 | Agrin |
| (SEQ ID NO:11) | (basal membrane component (lung)) | |
| XCDSSVS | 1/40 | NEPH2 immunoglobulin domain |
| (SEQ ID NO:12) | | (protein-protein interaction molecule) |
| VVGSADG | 1/40 | NEPH1 immunoglobulin domain |
| (SEQ ID NO:13) | | (protein-protein interaction molecule) |
| ISTFARG | 1/40 | T-cell receptor alpha chain |
| (SEQ ID NO:14) | (cellular immune response) | |
| VGVEYRT | 1/40 | ICAM-5, Telencephalin |
| (SEQ ID NO:15) | (Intercellular adhesin molecule) | |
| NPGYWGN | 1/40 | Extracellular binding protein family 5 |
| (SEQ ID NO:16) | | (Intercellular adhesin molecule) |

A “targeting peptide” as used herein is a peptide comprising a contiguous sequence of amino acids, which is characterized by selective association with an organism or cell of interest. Selective association may be determined, for example, by methods disclosed below, wherein the putative targeting peptide sequence is incorporated into a protein that is displayed on the outer surface of a phage. Exposure of an organism to a library of such phage that have been genetically engineered to express a multitude of such targeting peptides of different amino acid sequence is followed by collection of the organism, or one or more organs, tissues or cell types associated with the presence of the organism, which are typically derived from a subject, and identification of phage found associated with one or more cells or organisms. A phage expressing a targeting peptide sequence is considered to be selectively associated with an organism if it exhibits greater binding to that organism or cell as compared to a control organism or cell. Preferably, selective association of a targeting peptide should result in a two-fold or higher enrichment of the phage or peptide associated with the target organism or cell, compared to a control organism or cell. Selective association resulting in at least a three-fold, four-fold, five-fold, six-fold, seven-fold, eight-fold, nine-fold, ten-fold or higher enrichment of phage associated with the target organism(s) compared to a control organism is more preferred.

Alternatively, a phage expressing a targeting peptide sequence that exhibits selective association may be put in contact with a second organism or cell population for another round of screening. The second organism or cell population may be the same or different species or genus as the first organism or cell population screened against. Further enrichment may be exhibited following a third, fourth or more rounds of screening. “Targeting peptide” and “homing peptide” are used synonymously herein. In addition, host ligand is used to denote a peptide sequence in a protein of host to which a fungal cell binds.

In another embodiment, a subject with a fungal infection (e.g., IA) may be treated with a peptide, peptide conjugate, or peptidomimetic drug developed from screening a library using the BRASIL method. The subject may be treated with one or more peptidomimetic drug depending on the situation. In one embodiment, a subject with a fungal infection may be treated with a peptide compositions and/or peptidomimetic drug developed from screening a library using a selection process by at least one of the following routes including inhalation, intravenously, intraperitoneally, subcutaneously, intradermally, intranodally, intramuscularly, intranasally, orally, rectally, intravaginally, intravesicularly, intraocularly, and topically. In certain embodiments, a subject with a fungal infection (e.g., IA) may be treated with a peptide and/or peptidomimetic derivative by aerosolization and/or inhalation.

Other uses for a targeting peptide and/or peptidomimetic drug may be for identifying infection sites in an individual. For example, a targeting peptide that binds selectively or specifically to the surface of Aspergillus fumigatus and/or other fungi may be developed, using the disclosed methods. The anti-fungal targeting peptide may be labeled with a contrast or other imaging agent and introduced to subject or surface suspected of harboring a fungus of interest. The individual's lungs or other tissues may then be imaged with an imaging device. If an infected area is identified then this region may be specifically targeted by medical or a combined medical and surgical treatment. More specifically, if an area in the lung is identified early as the site of early sub-clinical IA, then this region could be targeted by antiifungals that combine with a peptide of the invention and/or a ligand-derived peptidomimetic drug(s). The ability to identify a specific region of early infection (e.g., in a specific area of the lung) may enable one of skill in the art to remove the exact region of infection by biopsy and/or specifically target the region for treatment. Also, for example, radiolabeled peptides could be used in order to diagnose the early phases of the invasion of a fungal organism (e.g., Aspergillus hyphae) in the lung parenchyma. Radiolabeling the GGRLGPF (the motif found more commonly from Aspergillus hyphae) could provide a quick means to diagnose the extent of the invasion of hyphae in the lungs (localized vs. widespread infection).

Other clinical entities exist where a peptide of the invention and/or a peptidomimetic drug may be useful. In certain embodiments, the targeting peptide of the invention and/or a ligand derived peptidomimetic drug may be used as a prophylactic against attachment of the fungal organism to a patient, an animal, an environmentally sensitive area, or an agricultural product such as a corn or other crop plant or product. In one embodiment, patients with pulmonary cavities (e.g., tuberculosis, cancer) who are at risk for aspergillomas may be treated with a peptide(s) alone or in combination with other treatments (including antifungal coupled to the peptide) to prevent inhaled Aspergillus conidia from attaching to the these cavities. The peptides may be selected to bind to surface proteins on the Aspergillus or alternatively to bind to tissues, organs, or receptors to which Aspergillus binds. In another embodiment, Allergic Bronchopulmonary Aspergillosis (ABPA) in patients with asthma may be treated with a peptide(s) alone or in combination with other treatments to prevent inhaled Aspergillus conidia from attaching to a host molecule causing a severe allergic reaction. In another embodiment, Fungal sinusitis in immunocompromised patients with cancer (leukemia/BMT) may be treated with a peptide(s) alone or in combination with other treatments described herein to prevent the development of sinusitis that is similar to pneumonia. In one embodiment, the peptide used as a treatment in any of the previous examples may be conida-derived WGHSRDE (SEQ ID NO: 1) and/or hyphae-derived GGRLGPF (SEQ ID NO: 8). Other than immunosuppressed patients (non-immunosuppressed patients such as asthma patients) any of these treatments mentioned above either alone (peptide treatment alone) or in combination (e.g., antifungal treatment(s)) may be used to prevent infection or prevent exacerbation of allergic sinusitis, ABPA and/or asthma. In other embodiments, peptides of the invention may be coupled to a solid surface, such as a filter, mask, or peptide array for capture of the fungus of interest.

In certain embodiments of the invention, it is contemplated that an identified peptide or ligand may be used to generate a peptide that is used to deliver an anti-fungal agent to the site of the infection. Methods of linking a peptide to an antifungal agent are known in the art. One method may be cross-linking the peptide to the anti-fungal agent and delivering the complex to a subject with a fungal infection. Another method includes the use of liposomes that harbor the antifungal agent and are linked to a peptide for targeting the complex to the affected area. Alternatively, slow-release microspheres may be coated with a peptide and the microspheres contain an anti-fungal agent to be released over a specified period of time. Thus, a sustained release of an anti-fungal agent in the target region alleviates repeated treatments and unnecessary exposure to high doses of an antifungal agent.

I. Fungal Pathogens

Systemic fungal infections cause approximately 25% of infection-related deaths in leukaemics. Infections due to Candida species are the fourth most important cause of nosocomial bloodstream infection. In certain other circumstances, fungal infections are also a major problem. Serious fungal infections may cause 5-10% of deaths in those undergoing lung, pancreas or liver transplantation. Acquired fungal sepsis occurs in up to 13% of very low birthweight infants.

There is a strong suggestion that invasive fungal infections have become more common in recent years, with a nearly 500% increase in the incidence of blood-stream infection with Candida spp. since the 1980s (Pfaller, 1995). Most systemic fungal infections are in fact due to Candida, but Aspergillus infections are also seen. Other causative organisms are less common, although in people with AIDS, a host of different fungi are important causes of disease and death—for example, Cryptococcus neoformans, as well as Histoplasma capsulatum, and strange organisms such as Pneumocystis carinii (considered by some a fungus), Penicillium marneffei, trichosporonosis, and fusariosis to name a few. Fungal infections in humans include, but are not limited to aspergillosis, blastomycosis, candidiasis, coccidioidomycosis, cryptococcosis, histoplasmosis, paracoccidiomycosis, sporotrichosis, or zygomycosis to name a few.

Studies of Aspergillus pathogenesis have used, with limited success, single gene knockouts (Chaveroche et al., 2000). More comprehensive strategies that exploit the differences in host receptor affinity between conidia and hyphae are needed. One strategy may be to identify specific host ligand sequences, or related peptide sequences, that bind to the surface of the pathogen(s). This approach is not limited to studying Aspergillus species but may also include other molds, for example, Fusarium species, Zygomycetes species and Scedosporium species.

A. Aspergillus

Aspergillus fumigatus is the most common species causing invasive aspergillosis (IA) (Latge, 1999). IA has emerged as one of the leading causes of death in patients with leukemia and bone marrow transplantation (Kontoyiannis and Bodey, 2002). The survival rate is poor, in agreement with the limited success of modern antifungals against Aspergillus species in immunosuppressed patient populations.

There are typically two stages of growth in Aspergillus species, conidia and hyphae. In healthy subjects, defense against inhaled Aspergillus fumigatus conidia, as well as other fungi, includes phagocytosis by macrophages. Initially, the resident lung macrophages are responsible for phagocytosis and, primarily, nonoxidative killing of the majority if not all the conidia (Clemons et al., 2000). Still, some conidia may escape phagocytosis, germinate to hyphae, and establish an invasive infection. If an infection is established, neutrophils are chemotactically attracted to the hyphae. Then these hyphal elements are subsequently destroyed extracellularly by the oxidative cytotoxic mechanisms of polymorphonuclear leukocytes (PMNs) (Latge, 1999, Clemons et al., 2000). Alternatively, in patients deficient in macrophage and PMN function are at high risk for the acquisition of IA (e.g., patients with neutropenia or prolonged use of corticosteroids), (Balow et al., 1975). These patients are in need of a targeted treatment for increasing their survivability. Also, patients predisposed to Aspergillus-conidia-induced allergic manifestations (e.g., patients with ABPA) could greatly benefit from approaches that prevent the attachment of conidia to the tissue of the respiratory tract.

B. Antifungal Agents

Common anti-fungal agents used to inhibit or treat fungal infections (e.g., Aspergillosis) are often ineffective against infections in affected subjects especially immunocompromised subjects. The effects of these agents are often that they fail to completely eradicate the infection caused by fungal organisms. In addition, once the lungs are infected the organism becomes difficult to target. In one embodiment of the invention, anti-fungal agents may be operatively coupled to or used in combination with identified peptides and/or host ligands that bind to surface components of a fungal organism (e.g., the surface of Aspergillus fumigatus conidia or hyphae). Examples of anti-fungal agents include but are not limited to polyenes (Amphotericin B formulations), triazoles (Itraconazole, voriconazole and the investigational posaconazole), Echinocandins (caspofungin and other investigational echinocandins) and Terbinafine. In one embodiment, an anti-fungal agent(s) may be introduced first (to alleviate the symptoms of the infection) and second a peptide, which may or may not be derived from an identified host ligand, may be introduced to attack the remaining pathogens or to direct a therapeutic to the proximity of the fungal cells. Alternatively, the treatments may be reversed providing a peptide treatment, with or without a coupled therapeutic, first followed by an anti-fungal agent(s) or the treatments may occur simultaneously depending on the patient and the level of infection.

Caspofungin (CAS) is a novel antifungal agent that irreversibly inhibits the enzyme 1,3-β-D-glucan synthase, preventing the formation of glucan polymers and disrupting the integrity of the fungal cell wall (Bowman et al., 2002). This glucan synthase complex is located in the apical tips of Aspergillus hyphae (Beauvais et al., 2001).

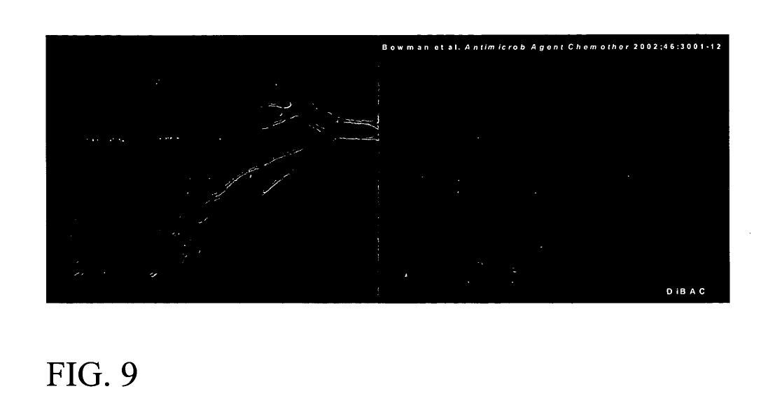

CAS has been recently approved for use in the treatment of IA in patients refractory to or intolerant of other therapies. CAS has shown efficacy in animal models of IA (Abruzzo et al., 1997), and liquid broth microdilution assays have demonstrated Aspergillus growth reduction resulting from CAS in vitro (Espinel-Ingroff, 1998). Furthermore, it was recently shown (by staining with the fluorescent dye DiBAC that allows visualization of non-viable compartments of fungi, FIG. 9) that CAS kills the apical tips of hyphae of growing A. fumigatus (Bowman et al., 2002). CAS-treated Aspergillus hyphae were also shown to be distorted and abnormal (Bowman, 2002). It is postulated that CAS might induce such alterations on the cell surface of A. fumigatus (especially in the growing apical hyphal tips) that could subsequently render that fungus vulnerable to the effector phagocytic cells of the immune system. In one embodiment, CAS may be introduced first (alleviate the symptoms of the infection) and second a peptide derived from an identified host ligand (likely prescreened for attachment to a CAS-exposed organism) may be introduced to attack the remaining pathogens. Alternatively, the treatments may be reversed providing a peptide treatment first followed by CAS treatment or the treatments may occur simultaneously depending on the condition of the patient and the level of infection. In addition, CAS may be operatively coupled to a targeting peptide.

In other embodiments, a therapeutic coupled to or use in conjunction with a targeting peptide is an antifungal agent. Some exemplary classes of antifungal agents include imidazoles or triazoles such as clotrimazole, miconazole, ketoconazole, econazole, butoconazole, omoconazole, oxiconazole, terconazole, itraconazole, fluconazole, voriconazole (UK 109,496), posaconazole, ravuconazole or flutrimazole; the polyene antifungals such as amphotericin B, liposomal amphoterecin B, natamycin, nystatin and nystatin lipid formualtions; the cell wall active cyclic lipopeptide antifungals, including the echinocandins such as caspofungin, micaflngin, anidulfungin, cilofungin; LY121019; LY303366; the allylamine group of antifungals such as terbinafine. Yet other non-limiting examples of antifungal agents include naftifine, tolnaftate, mediocidin, candicidin, trichomycin, hamycin, aurefungin, ascosin, ayfattin, an azole, terbinafine, azacolutin, trichomycin, levorin, heptamycin, candimycin, griseofulvin, BF-796, MTCH 24, BTG-137586, pradimicins (MNS 18184), benanomicin; ambisome; nikkomycin Z; flucytosine, or perimycin.

II. Identification of a Targeting Peptides

The invention comprises methods for the identification of one or more targeting peptides or molecular targets that could be utilized for the development of novel therapies to treat fungal infections. Employing Biopanning and Rapid Analysis of Selective Interactive Ligands (BRASIL), fungal cells are profiled, including, but not limited to Aspergillus. Screening of the fungal cells, including Aspergillus cells, with CXₙC, wherein in can be 4, 5, 6, 7, or more residues, random phage library that yield several peptide motifs. In one example, clones (encoding SEQ ID NO: 1 to SEQ ID NO: 16) exhibited high frequency binding to various fungal cells as compared to the control insert-less phage. Comparison of the selected motifs with available sequences in on-line protein databases suggests that a number of candidate proteins share homologous sequences with these peptides. The findings will also have important clinical implications in that newly identified motifs may serve as a peptidomimetic drug leads and can be optimized as delivery vehicles for targeted therapy of fungal infections.

BRASIL has been successfully used to isolate phage in various cell systems such as activated endothelial cells and tumor cells. BRASIL has also been used to isolate bone marrow homing phage using in vivo/ex-vivo based strategies. To identify peptides that bind fungal cells, fungal cells are incubated with peptide encoding phage or control phage. Phage bound to the cells are recovered, and quantified.

Once novel targeting peptides and/or host ligands are identified against the fungal organisms (e.g., by screening a phage display library), they may be used to develop peptidomimetic drugs and/or used for therapeutic or diagnostic purposes. In one embodiment, a subject with a fungal infection (e.g., IA) may be treated with a peptidomimetic drug developed from screening a library using a selection process as described herein or is known to those of skill in the art.

A. Phage Display

Recently, an in vivo selection system was developed using phage display libraries to identify organ, tissue or cell type-targeting peptides in a mouse model system. Phage display libraries expressing transgenic peptides on the surface of bacteriophage were initially developed to map epitope binding sites of immunoglobulins (Smith and Scott, 1985 and 1993). Such libraries can be generated by inserting random oligonucleotides into cDNAs encoding a phage surface protein, and generating collections of phage particles displaying unique peptides in as many as 10⁹ permutations (Pasqualini and Ruoslahti, 1996; Arap et al., 1998a and 1998b).

A “phage display library” is a collection of phage that have been genetically engineered to express a set of putative targeting peptides on their outer surface. In preferred embodiments, DNA sequences encoding the putative targeting peptides are inserted in frame into a gene encoding a phage capsule protein. In other preferred embodiments, the putative targeting peptide sequences are in part random mixtures of all twenty amino acids and in part non-random. In certain preferred embodiments the putative targeting peptides of the phage display library exhibit one or more cysteine residues at fixed locations within the targeting peptide sequence. Cysteines may be used, for example, to create a cyclic peptide.

B. Biopanning and Rapid Analysis of Selective Interactive Ligands (BRASIL)

In preferred embodiments, separation of phage bound to the cells of a target organisms or cells from unbound phage is achieved using the BRASIL (Biopanning and Rapid Analysis of Soluble Interactive Ligands) technique (PCT Application PCT/US01/28124 entitled, “Biopanning and Rapid Analysis of Selective Interactive Ligands (BRASIL)” by Arap et al., filed Sep. 7, 2001, incorporated herein by reference in its entirety). In BRASIL, an organ, tissue or cell is gently separated into cells or small clumps of cells that are suspended in an aqueous phase. The aqueous phase is layered over an organic phase of appropriate density and centrifuged. Cells attached to bound phage are pelleted at the bottom of the centrifuge tube, while unbound phage remain in the aqueous phase. This allows a more efficient separation of bound from unbound phage, while maintaining the binding interaction between phage and cell. BRASIL may be performed in an in vivo protocol, in which organs, tissues or cell types are exposed to a phage display library by intravenous administration, or by an ex vivo protocol, where the cells are exposed to the phage library in the aqueous phase before centrifugation.

C. Preparation of Large Scale Primary Libraries

n certain embodiments, primary phage libraries are amplified before injection into a human subject. A phage library is prepared by ligating targeting peptide-encoding sequences into a phage vector, such as fUSE5. The vector is transformed into pilus negative host E. coli such as strain MC1061. The bacteria are grown overnight and then aliquots are frozen to provide stock for library production. Use of pilus negative bacteria avoids the bias in libraries that arises from differential infection of pilus positive bacteria by different targeting peptide sequences.

To freeze, bacteria are pelleted from two thirds of a primary library culture (5 liters) at 4000×g for 10 min. Bacteria are resuspended and washed twice with 500 ml of 10% glycerol in water, then frozen in an ethanol/dry ice bath and stored at −80° C.

For amplification, 1.5 ml of frozen bacteria are inoculated into 5 liters of LB medium with 20 μg/ml tetracycline and grown overnight. Thirty minutes after inoculation, a serial dilution is plated on LB/tet plates to verify the viability of the culture. If the number of viable bacteria is less than 5-10 times the number of individual clones in the library (1-2×10⁸) the culture is discarded.

After growing the bacterial culture overnight, phage are precipitated. About ¼ to ⅓ of the bacterial culture is kept growing overnight in 5 liters of fresh medium and the cycle is repeated up to 5 times. Phage are pooled from all cycles and used for injection into human subjects.

Intravenous administration of phage display libraries to mice was followed by the recovery of phage from individual organs (Pasqualini and Ruoslahti, 1996). Phage were recovered that were capable of selective homing to the vascular beds of different mouse organs, tissues or cell types, based on the specific targeting peptide sequences expressed on the outer surface of the phage (Pasqualini and Ruoslahti, 1996). A variety of organ and tumor-homing peptides have been identified by this method (Rajotte et al., 1998; Rajotte et al., 1999; Koivunen et al., 1999a; Burg et al., 1999; Pasqualini, 1999). Each of those targeting peptides bound to different receptors that were selectively expressed on the vasculature of the mouse target tissue (Pasqualini, 1999; Pasqualini et al., 2000; Folkman, 1997; Folkman, 1995). Tumor-homing peptides bound to receptors that were upregulated in the tumor angiogenic vasculature of mice (Brooks et al., 1994b; Pasqualini et al., 2000). In addition to identifying individual targeting peptides selective for an organ, tissue or cell type (Pasqualini and Ruoslahti, 1996; Arap et al., 1998a; Koivunen et al., 1999b), this system has been used to identify endothelial cell surface markers that are expressed in mice in vivo (Rajotte and Ruoslahti, 1999).

Attachment of therapeutic agents to targeting peptides resulted in the selective delivery of the agent to a desired organ, tissue or cell type in the mouse model system. Targeted delivery of chemotherapeutic agents and proapoptotic peptides to receptors located in tumor angiogenic vasculature resulted in a marked increase in therapeutic efficacy and a decrease in systemic toxicity in tumor bearing mouse models (Arap et al., 1998a, 1998b; Ellerby et al., 1999).

The methods described herein for identification of targeting peptides involve the in vivo administration of phage display libraries. Various methods of phage display and methods for producing diverse populations of peptides are well known in the art. For example, U.S. Pat. Nos. 5,223,409; 5,622,699 and 6,068,829, each of which is incorporated herein by reference in its entirety, disclose methods for preparing a phage library. The phage display technique involves genetically manipulating bacteriophage so that small peptides can be expressed on their surface (Smith and Scott, 1985 and 1993). The potential range of applications for this technique is quite broad, and the past decade has seen considerable progress in the construction of phage-displayed peptide libraries and in the development of screening methods in which the libraries are used to isolate peptide ligands. For example, the use of peptide libraries has made it possible to characterize interacting sites and receptor-ligand binding motifs within many proteins, such as antibodies involved in inflammatory reactions or integrins that mediate cellular adherence. This method has also been used to identify novel peptide ligands that serve as leads to the development of peptidomimetic drugs or imaging agents (Arap et al., 1998a). In addition to peptides, larger protein domains such as single-chain antibodies can also be displayed on the surface of phage particles (Arap et al., 1998a).

Targeting peptides selective for a given organ, tissue or cell type can be isolated by “biopanning” (Pasqualini and Ruoslahti, 1996; Pasqualini, 1999). In brief, a library of phage containing putative targeting peptides is administered to an animal or human and samples of organs, tissues or cell types containing phage are collected. In preferred embodiments utilizing filamentous phage, the phage may be propagated in vitro between rounds of biopanning in pilus-positive bacteria. The bacteria are not lysed by the phage but rather secrete multiple copies of phage that display a particular insert. Phage that bind to a target molecule can be eluted from the target organism, organ, tissue or cell type and then amplified by growing them in host bacteria. If desired, the amplified phage can be administered to a host and samples of organs, tissues or cell types again collected. Multiple rounds of biopanning can be performed until a population of selective binders is obtained. The amino acid sequence of the peptides is determined by sequencing the DNA corresponding to the targeting peptide insert in the phage genome. The identified targeting peptide can then be produced as a synthetic peptide by standard protein chemistry techniques (Arap et al., 1998a, Smith and Scott, 1985). This approach allows circulating targeting peptides to be detected in an unbiased functional assay, without any preconceived notions about the nature of their target. Once a candidate target is identified as the receptor of a targeting peptide, it can be isolated, purified and cloned by using standard biochemical methods (Pasqualini, 1999; Rajotte and Ruoslahti, 1999).

In certain embodiments, a subtraction protocol is used may be used to further reduce background phage binding. The purpose of subtraction is to remove phage from the library that bind to cells other than the cell of interest, that bind to inactivated cells, or bind either conidia or hyphae without binding the other. In alternative embodiments, the phage library may be prescreened against a subject who does not possess the targeted cell, tissue or organ. For example, placenta-binding peptides may be identified after prescreening a library against a male or non-pregnant female subject. After subtraction the library may be screened against the cell, tissue or organ of interest. In another alternative embodiment, an unstimulated, quiescent cell type, tissue or organ may be screened against the library and binding phage removed. The cell line, tissue or organ is then activated, for example by administration of a hormone, growth factor, cytokine or chemokine and the activated cell type, tissue or organ screened against the subtracted phage library. Other subtraction protocols are known and may be used in the practice of the present invention, for example as disclosed in U.S. Pat. Nos. 5,840,841, 5,705,610, 5,670,312 and 5,492,807, which are incorporated herein by reference in their entirety.

D. Choice of Phage Display System

Previous in vivo selection studies performed in mice preferentially employed libraries of random peptides expressed as fusion proteins with the gene III capsule protein in the fUSE5 vector (Pasqualini and Ruoslahti, 1996). The number and diversity of individual clones present in a given library is a significant factor for the success of in vivo selection. It is preferred to use primary libraries, which are less likely to have an over-representation of defective phage clones (Koivunen et al., 1999b). The preparation of a library should be optimized to between 10⁸-10⁹ transducing units (T.U.)/ml. In certain embodiments, a bulk amplification strategy is applied between each round of selection.

Phage libraries displaying linear, cyclic, or double cyclic peptides may be used within the scope of the present invention. However, phage libraries displaying 3 to 10 random residues in a cyclic insert (CX₃₋₁₀C) are preferred, since single cyclic peptides tend to have a higher affinity for the target organ than linear peptides. Libraries displaying double-cyclic peptides (such as CX₃C X₃CX₃C; Rajotte et al., 1998) have been successfully used. However, the production of the cognate synthetic peptides, although possible, can be complex due to the multiple conformers with different disulfide bridge arrangements.

III. Targeted Delivery

Embodiments of the invention include methods for introducing a treatment or diagnostic agent. In certain embodiments, the peptides or proteins of the present invention may be attached to therapeutic agents and/or imaging agents of use for treatment, imaging, and diagnosis of various diseased organs or tissues (e.g., fungal infections). Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

In particular embodiments the treatment agent is in a sustained release composition. The preferred period for sustained release of one or more agents is for a period of one to twelve weeks, preferably two to eight weeks. Methods for local delivery of sustained release agents include but are not limited to an aspirator for oral application, direct application, injection, or using other methods as indicated herein or in the art.

One embodiment of a composition suitable for the described method includes the use of a bioerodiable microparticle harboring one or more of the aforementioned antifungal agents and/or ligand derived peptide (i.e., targeting peptide or its derivative). The bioerodible microparticle may consist of a bioerodible polymer such as poly (lactide-co-glycolide). The composition of the bioerodible polymer is controlled to release the agent over a period of 1-2 weeks. It was previously demonstrated that biodegradable microparticles, for example, poly(lactide-co-glycolide) were capable of controlled release of an oligonucleotide. These microparticles were prepared by the multiple emulsion-solvent evaporation technique. In order to increase the uptake of the oligonucleotide into the microparticles it was accompanied by polyethylenimine (PEI). The PEI also tended to make the microparticles more porous thus facilitating the delivery of the oligonucleotide out of the particles (see De Rosa et al., 2002). In one preferred embodiment of a composition, the bioerodible microparticle may be a PLGA polymer 50:50 with carboxylic acid end groups. PLGA is a base polymer often used for controlled release of drugs (i.e., anti-cancer drugs such as anti-prostate cancer agents). Two common delivery forms for controlled release include a microcapsule and a microparticle (e.g., a microsphere). The polymer and the agent are combined and usually heated to form the microparticle prior to delivery to the site of interest (Mitsui Chemicals, Inc). As the microparticles erode a porous network of the microparticle composition is formed in the affected region resulting in a matrix with a controlled pore size. As the porous network is formed at least one antifungal and/or peptidomimetic agent (e.g., CAS anti-fungal and/or WGHSRDE peptide of Aspergillus) may be released. One embodiment includes a peptide-coated bioerodible polymer harboring the anti-microbial agent CAS. In one embodiment, the PLGA polymer 50:50 with carboxylic acid end groups harbors CAS for slow release. It is preferred that each microparticle may release at least 20 percent of its contents and more preferably around 90 percent of its contents. In one embodiment, the microparticle harboring at least one anti-fungal and/or peptidomimetic will degrade slowly over time releasing the agent and/or factor or release the agent and/or factor immediately upon contact with the affected area in order to rapidly attack the pathogen (e.g., Aspergillus hyphae). In another embodiment, the microparticles may be a combination of controlled-release microparticles and immediate release microparticles. A preferred rate of deposition of the delivered factor will vary depending on the condition of the subject undergoing treatment and extent of the infection. It is also contemplated that non-bioerodible particles may be used to deliver an antifungal and/or peptide (e.g., CAS antifungal and or WGHSRDE peptide of Aspergillus conidia). The non-bioerodible microparticle may consist of a non-bioerodible polymer such as an acrylic based microsphere for example a tris acryl microsphere (Biosphere Medical).

In one embodiment, the treatment agent compositions suitable for treatment of the infected zone are rendered resistant to phagocytosis by inhibiting opsonin protein absorption of the particles. In this regard, treatment agent compositions including sustained release carriers include particles having an average diameter up to about 10 microns are considered. In other situations, the particle size may range from about 1 mm to about 200 mm. The larger size particles may be considered in certain cases to avoid macrophage frustration and to avoid chronic inflammation in the treatment site.

IV. Proteins and Peptides

In certain embodiments, the present invention concerns novel compositions comprising at least one protein or peptide. As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide” and “peptide are used interchangeably herein.

In certain embodiments the size of at least one protein or peptide may comprise, but is not limited to, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties. Accordingly, the term protein or peptide encompasses amino acid sequences comprising at least one of the 20 common amino acids found in naturally occurring proteins, or at least one modified or unusual amino acid, including, but not limited to, 2 Aminoadipic acid (Aad), N Ethylasparagine (EtAsn), 3 Aminoadipic acid (Baad), Hydroxylysine (Hyl), β alanine, β Amino propionic acid (Bala), allo Hydroxylysine (AHyl), 2 Aminobutyric acid (Abu), 3 Hydroxyproline (3Hyp), 4 Aminobutyric acid (4Abu), 4 Hydroxyproline (4Hyp), 6 Aminocaproic acid (Acp), Isodesmosine (Ide), 2 Aminoheptanoic acid (Ahe), allo Isoleucine (AIle), 2 Aminoisobutyric acid (Aib), N Methylglycine (MeGly), 3 Aminoisobutyric acid (Baib), N Methylisoleucine (MeIle), 2 Aminopimelic acid (Apm), 6 N Methyllysine (MeLys), 2,4 Diaminobutyric acid (Dbu), N Methylvaline (MeVal), Desmosine (Des), Norvaline (Nva), 2,2′Diaminopimelic acid (Dpm), Norleucine (Nle), 2,3 Diaminopropionic acid (Dpr), Ornithine (Orn), or N Ethylglycine (EtGly).

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. Coding regions for known genes may be amplified and/or expressed using the techniques disclosed herein or as would be know to those of ordinary skill in the art. Alternatively, various commercial preparations of proteins, polypeptides and peptides are known to those of skill in the art.

A. Peptide Mimetics

Another embodiment for the preparation of molecule or compound according to the invention is the use of peptide mimetics that mimic characteristics of all or part of the peptides identified herein. Mimetics are molecules that mimic elements of protein secondary structure (see, for example, Johnson et al., 1993, incorporated herein by reference). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting peptides disclosed herein, but with altered and even improved characteristics.

B. Fusion Proteins

Other embodiments of the present invention concern fusion proteins. These molecules generally have all or a substantial portion of a targeting peptide, linked at the N— or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the instant invention comprise a targeting peptide linked to a therapeutic protein or peptide. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually and protein or peptide could be incorporated into a fusion protein comprising a targeting peptide.

Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

C. Protein Purification

In certain embodiments a protein or peptide may be isolated or purified. Protein purification techniques are well known to those of skill in the art. These techniques involve, at one level, the homogenization and crude fractionation of the cells, tissue or organ to polypeptide and non-polypeptide fractions. The protein or peptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. A particularly efficient method of purifying peptides is fast performance liquid chromatography (FPLC) or even high performance liquid chromatography (HPLC).

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the protein or peptide in the composition.

Various methods for quantifying the degree of purification of the protein or peptide are known to those of skill in the art in light of the present disclosure. These include, for example, determining the specific activity of an active fraction, or assessing the amount of protein or peptide within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing some other chromatography systems. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, and temperature). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

D. Synthetic Peptides

Because of their relatively small size, the targeting peptides of the invention can be synthesized in solution or on a solid support in accordance with conventional techniques. Various automatic synthesizers are commercially available and can be used in accordance with known protocols (see, for example, Stewart and Young, 1984; Tam et al., 1983; Merrifield, 1986; or Barany and Merrifield, 1979, each incorporated herein by reference). Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

E. Antibodies

In certain embodiments, it may be desirable to make antibodies against the identified targeting peptides or their receptors. The appropriate targeting peptide or receptor, or portions thereof, may be coupled, bonded, bound, conjugated, or chemically-linked to one or more agents via linkers, polylinkers, or derivatized amino acids. This may be performed such that a bispecific or multivalent composition or vaccine is produced. It is further envisioned that the methods used in the preparation of these compositions are familiar to those of skill in the art and should be suitable for administration to humans, i.e., pharmaceutically acceptable. Preferred agents are the carriers are keyhole limpet hemocyanin (KLH) or bovine serum albumin (BSA).

The term “antibody” is used to refer to any antibody like molecule that has an antigen binding region, and includes antibody fragments such as Fab′, Fab, F(ab′)2, single domain antibodies (DABs), Fv, scFv (single chain Fv), and the like. Techniques for preparing and using various antibody based constructs and fragments are well known in the art. Means for preparing and characterizing antibodies are also well known in the art (See, e.g., Harlow and Lane, 1988; incorporated herein by reference).

F. Cytokines and Chemokines

In certain embodiments, it may be desirable to couple specific bioactive agents to one or more targeting peptides for targeted delivery to an area of fungal infection or growth. Such agents include, but are not limited to, cytokines and chemokines. The term “cytokine” is a generic term for proteins released by one cell population that act on another cell as intercellular mediators.

Examples of such cytokines are lymphokines, monokines, growth factors and traditional polypeptide hormones. Included among the cytokines are prostaglandin; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-10, IL-11, IL-12; IL-13, IL-14, IL-15, IL-16, IL-17, and IL-18. As used herein, the term cytokine includes proteins from natural sources or from recombinant cell culture and biologically active equivalents of the native sequence cytokines.

Chemokines generally act as chemoattractants to recruit immune effector cells to the site of chemokine expression. It may be advantageous to express or localize a particular chemokine gene in combination with, for example, a cytokine gene, to enhance the recruitment of other immune system components to the site of treatment. Chemokines include, but are not limited to, RANTES, MCAF, MIP1-alpha, MIP1-Beta, and IP-10. The skilled artisan will recognize that certain cytokines are also known to have chemoattractant effects and could also be classified under the term chemokines.

G. Imaging Agents and Radioisotopes

In certain embodiments, the claimed peptides or proteins of the present invention may be attached to imaging agents of use for imaging and diagnosis of various disease states. Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X ray imaging, include but are not limited to lanthanum (III), gold (III), lead (II), and especially bismuth (III).

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium^{188,} ⁷⁵selenium, ³⁵sulphur, technicium⁹⁹m and yttrium⁹⁰. ¹²⁵I is often being preferred for use in certain embodiments, and technicium⁹⁹m and indium¹¹¹ are also often preferred due to their low energy and suitability for long range detection.

Radioactively labeled proteins or peptides of the present invention may be produced according to well known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides according to the invention may be labeled with technetium^{99m} by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium potassium phthalate solution, and the peptide. Intermediary functional groups that are often used to bind radioisotopes that exist as metallic ions to peptides are diethylenetriaminepenta-acetic acid (DTPA) and ethylene diaminetetra-acetic acid (EDTA). Also contemplated for use are fluorescent labels, including rhodamine, fluorescein isothiocyanate and renographin.

In certain embodiments, the claimed proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

H. Cross-Linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. Nos. 5,603,872 and 5,401,511, each specifically incorporated herein by reference in its entirety. Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites are dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl)carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups.

V. Nucleic Acids

Nucleic acids according to the present invention may encode a targeting peptide, a receptor protein, a fusion protein, or other protein or peptide. The nucleic acid may be derived from genomic DNA, complementary DNA (cDNA) or synthetic DNA. Where incorporation into an expression vector is desired, the nucleic acid may also comprise a natural intron or an intron derived from another gene. Such engineered molecules are sometime referred to as “mini-genes.”

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of almost any size, determined in part by the length of the encoded protein or peptide.

It is contemplated that targeting peptides, fusion proteins and receptors may be encoded by any nucleic acid sequence that encodes the appropriate amino acid sequence. The design and production of nucleic acids encoding a desired amino acid sequence is well known to those of skill in the art, using standardized codon tables. In preferred embodiments, the codons selected for encoding each amino acid may be modified to optimize expression of the nucleic acid in the host cell of interest. Codon preferences for various species of host cell are well known in the art.

In addition to nucleic acids encoding the desired peptide or protein, the present invention encompasses complementary nucleic acids that hybridize under high stringency conditions with such coding nucleic acid sequences. High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

VI. Pharmaceutical Compositions

Where clinical applications are contemplated, it may be necessary to prepare pharmaceutical compositions—peptides, peptide conjugates, proteins, antibodies and drugs—in a form appropriate for the intended application. Generally, this will entail preparing compositions that are essentially free of impurities that could be harmful to humans or animals.

One generally will desire to employ appropriate salts and buffers to render delivery peptides stable and allow for uptake by target cells. Buffers also are employed when recombinant cells are introduced into a patient. Aqueous compositions of the present invention may comprise an effective amount of a protein, peptide, antibody, fusion protein, recombinant phage and/or expression vector, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the proteins or peptides of the present invention, its use in therapeutic compositions is contemplated. Supplementary active ingredients also can be incorporated into the compositions.

The active compositions of the present invention may include classic pharmaceutical preparations. Administration of these compositions according to the present invention are via any common route so long as the target tissue is available via that route. This includes oral, nasal, buccal, rectal, vaginal or topical. Alternatively, administration may be by aerosol, inhalation, orthotopic, intradermal, subcutaneous, intramuscular, intraperitoneal, intraarterial or intravenous injection. Such compositions normally would be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

VII. Additional Therapeutic Agents

In certain embodiments, therapeutic agents may be attached to a targeting peptide or fusion protein for selective delivery to fungal cells. Agents or factors suitable for use may include any chemical compound that induces apoptosis, cell death, and/or cell stasis. In addition to antifungals described above various other therapeutic agents may be used in conjunction with the present invention.

Certain antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of cytotoxic antibiotics include, but are not limited to, bleomycin, dactinomycin, daunorubicin, doxorubicin (Adriamycin), plicamycin (mithramycin) and idarubicin.

The skilled artisan is directed to “Remington's Pharmaceutical Sciences” 15th Edition, chapter 33, and in particular to pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA Office of Biologics standards.

VIII. Kits

In still further embodiments, the present invention concerns kits for use with the therapeutic and diagnostic methods described above. As the encoded proteins or peptides may be employed to target delivery of a therapeutic to a fungal cell, and/or to detect antibodies or the corresponding antibodies may be employed to detect encoded proteins or peptides, either or both of such components may be provided in the kit. The immunodetection kits will thus comprise, in suitable container means, an encoded protein or peptide, or a first antibody that binds to an encoded protein or peptide, and an immunodetection reagent.

In certain embodiments, the encoded protein or peptide, or the first antibody that binds to the encoded protein or peptide, may be bound to a solid support, such as a column matrix or well of a microtiter plate.

Immunodetection reagents of the kit may take any one of a variety of forms, including those detectable labels that are associated with or linked to the given antibody or antigen, and detectable labels that are associated with or attached to a secondary binding ligand. Exemplary secondary ligands are those secondary antibodies that have binding affinity for the first antibody or antigen, and secondary antibodies that have binding affinity for a human antibody.

Further suitable immunodetection reagents for use in the present kits include the two-component reagent that comprises a secondary antibody that has binding affinity for the first antibody or antigen, along with a third antibody that has binding affinity for the second antibody, the third antibody being linked to a detectable label.

The kits may further comprise a suitably aliquoted composition of the encoded protein or polypeptide, whether labeled or unlabeled, as may be used to prepare a standard curve for a detection assay.

The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the peptide, peptide conjugate, antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Library Screening of Aspergillus fumigatus

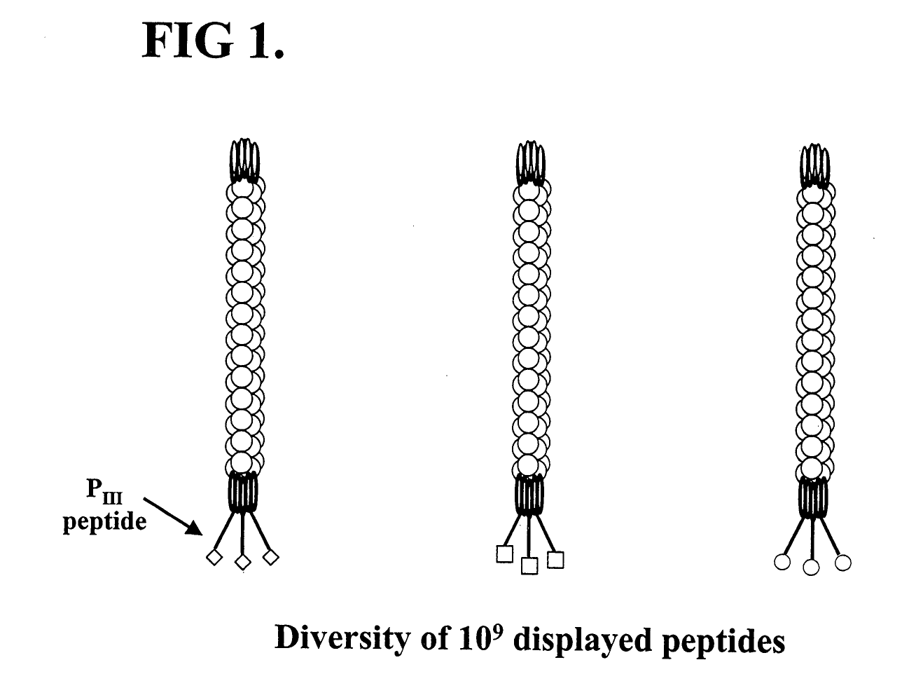

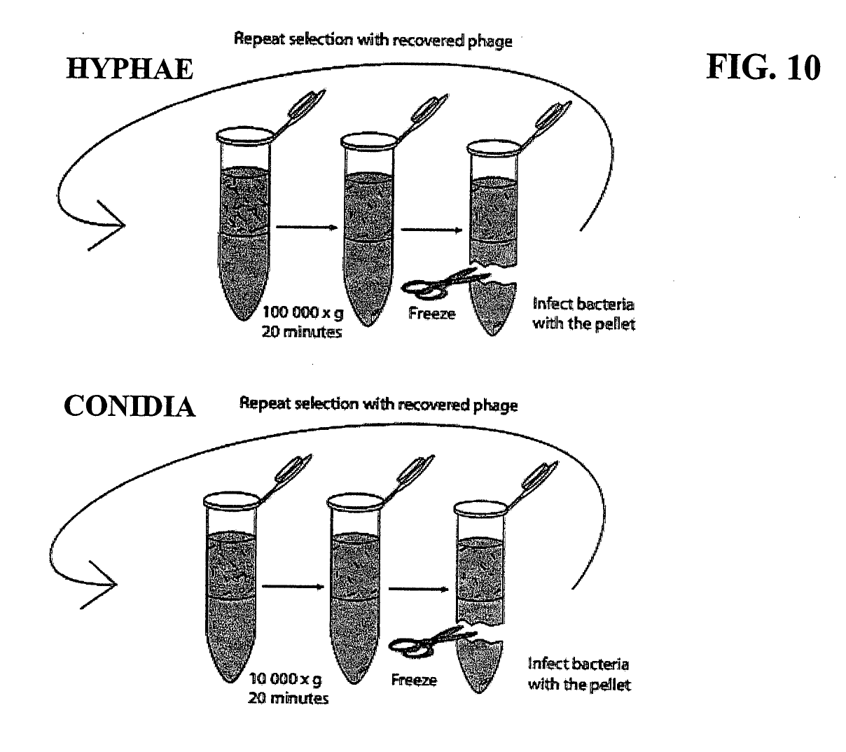

To identify potential host ligands that bind on the surface of A. fumigatus conidia and hyphae, a phage display library (CX₇C) for A. fumigatus conidia and hyphae binding ligands using BRASIL is screened. The Aspergillus fumigatus clinical isolate AF293 (currently being used in the Aspergillus sequencing project, provided kindly by Dr. D. Denning, Manchester, UK.) is plated on YAG plates at 37° C. for 3 days and conidia are collected. Conidia (suspensions of 10⁴ conidia/ml) are incubated at 37° C. for 16-20 hours in liquid YAG medium to allow for germination to hyphae. Conidia or hyphae, collected as described earlier, were then incubated with 10⁹ transducing units (TU) of CX₇C phage display library (Arap et al., 2002). A phage-conidia/hyphae suspension in an upper aqueous phase is centrifuged through a non-miscible organic phase with an intermediate specific density. Upon optimized centrifugation conditions conidia or hyphae entered the lower organic phase and pellet at the bottom of the tube, carrying with them only the specifically bound phage (FIG. 10). Dibutylphtalate:Cyclohexane ratio of the organic phase is preferably 6:1. Hyphae needed 100,000 g for pelleting as compared to 10,000 g for conidia (FIG. 10). The conidia/hyphae pellet is then recovered (after freezing, FIG. 10), and the phage is rescued by infection of E. coli K91kan bacteria (FIG. 1). Amplified phage was then used for subsequent rounds of selection (FIG. 10)

Infected E. coli K91Kan were incubated overnight in LB medium with kanamycin as selection marker (70 μg/ml), tetracycline that served as inducer of phage multiplication (40 μg/ml, the phage also contains the tetracycline resistance gene tetR), and voriconazole for suppression of conidial and hyphal growth (4 μg/ml). The next day, the phage was first separated from E. coli by centrifuigation, recovered using PEG/NaCl, and finally tittrated (to 109 TU units/μl) in order to be used in subsequent selection rounds.

After 4 rounds of selection for conidia 5 rounds for hyphae, peptide-inserts of 40 random phage clones (each for conidia and hyphae) were sequenced and analyzed for peptide sequences according to enrichment and by ClustalW sequence alignment. Selected motifs were then used to search non-redundant protein databanks.

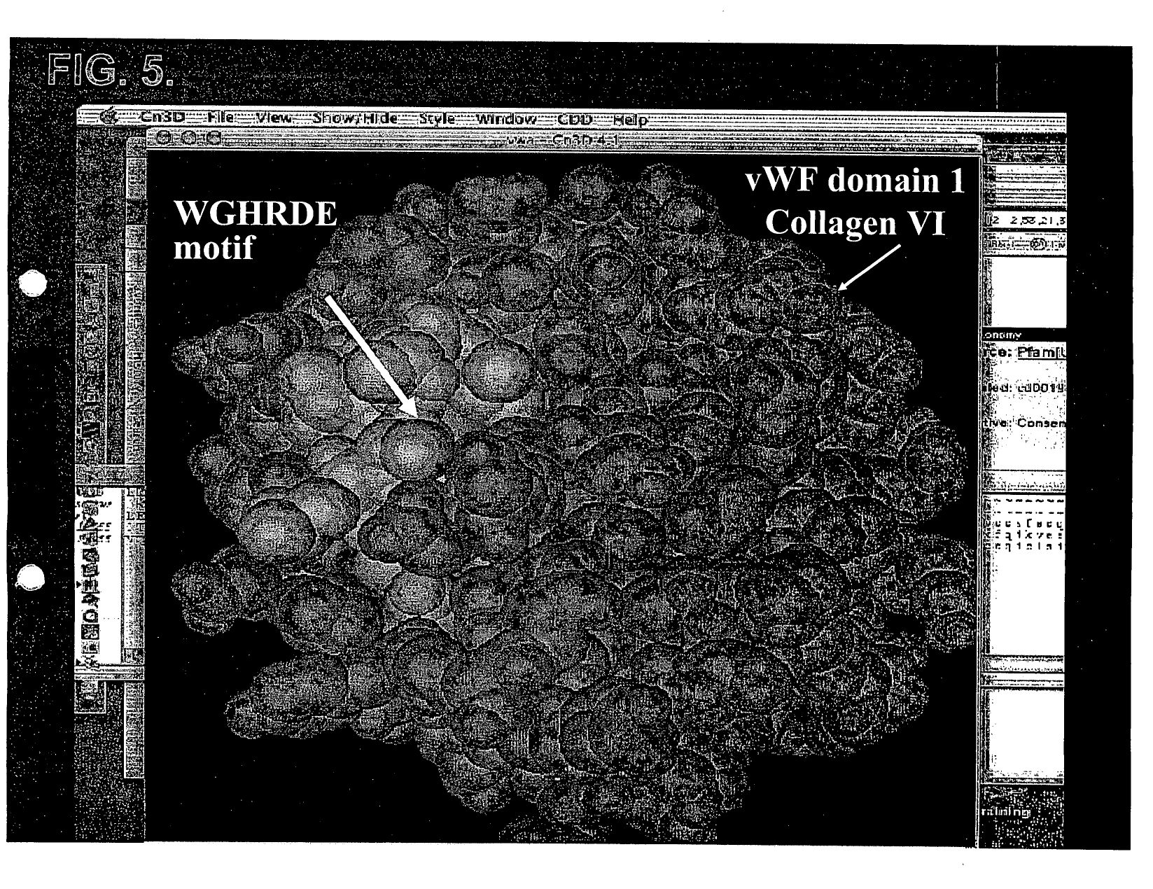

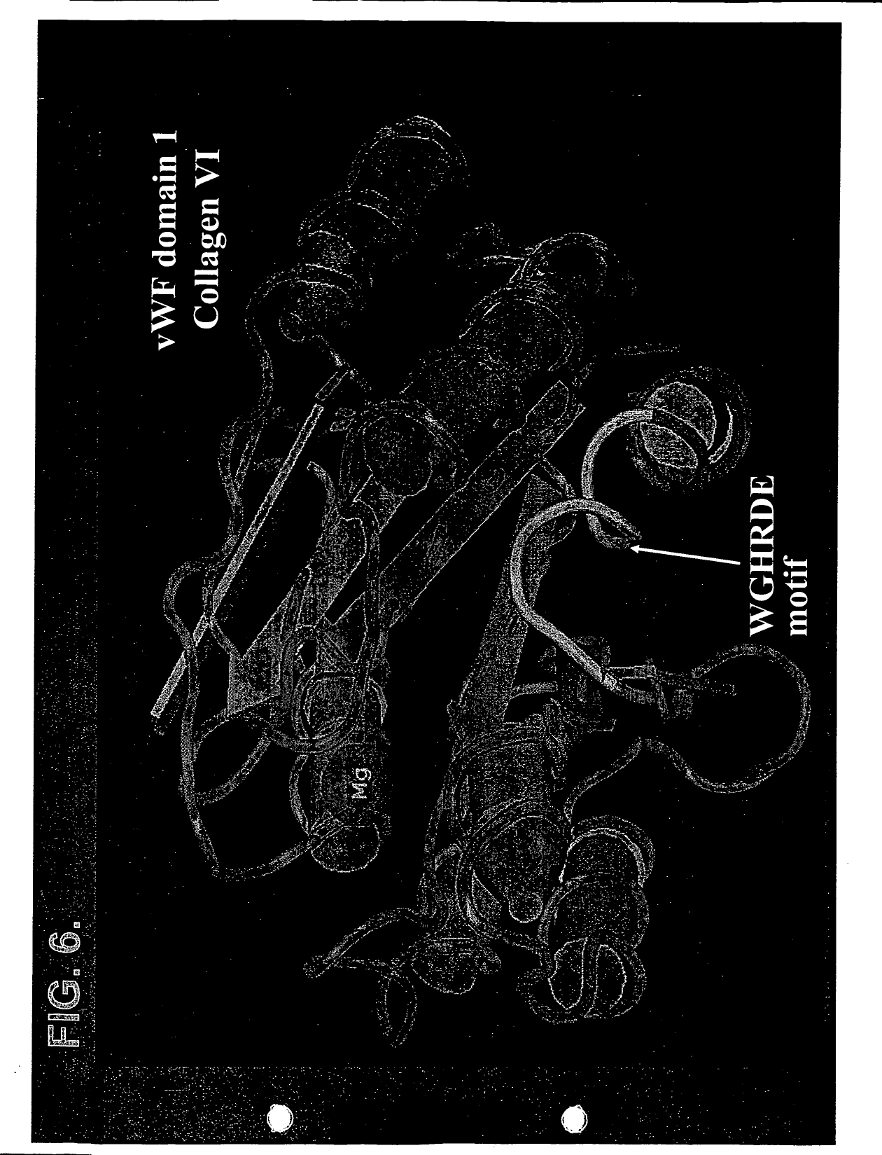

In one observation, a repeated peptide motif with high similarity to collagen VI as potential ligand on the conidial surface was observed. Among these motifs, the most commonly recovered WGHSRDE (SEQ ID NO:1) motif was found to be located on the surface of the vWF domain 1 of collagen VI (FIGS. 5 and 6). A greater diversity in peptide motifs was observed in hyphae-binding ligands. The motif most frequently found as potential ligand on the hyphal surface had similarity with several other molecules of the extracellular matrix (collagen I-V, IX-XI, laminin V, thrombospondin I-V, basilin, agrin), and macrophage and T-cell receptors. The binding specificity of the most commonly recovered phages was confirmed as harboring peptides bound to ligands from conidia/hyphae using Fd-tet phage (an insertless phage) as control. The number of these phages recovered from conidia and hyphae was 50 and 100-fold higher than Fd-tet respectively.

To validate further the binding of the candidate human proteins to conidia or hyphae 125I-labeled proteins may be used. Commercially available candidate proteins will be labeled with Na125I using chloramine T-method generating gamma-emitting protein with specific activity of at least 0.1 mCi/mg. The inventors will incubate 1 μg (0.1 μCi) of candidate protein (or control protein) with conidia or hyphae at +4° C. for 4 hours and centrifuge the suspension through a non-miscible organic phase with an intermediate specific density. The pellet will be recovered, and the amount of bound protein in the pellet will be determined by counting the amount of ¹²⁵I-labeled protein in a gamma counter.

### Example 2

### BRASIL Method for CAS-Exposed Aspergillus Conidia and Hyphae

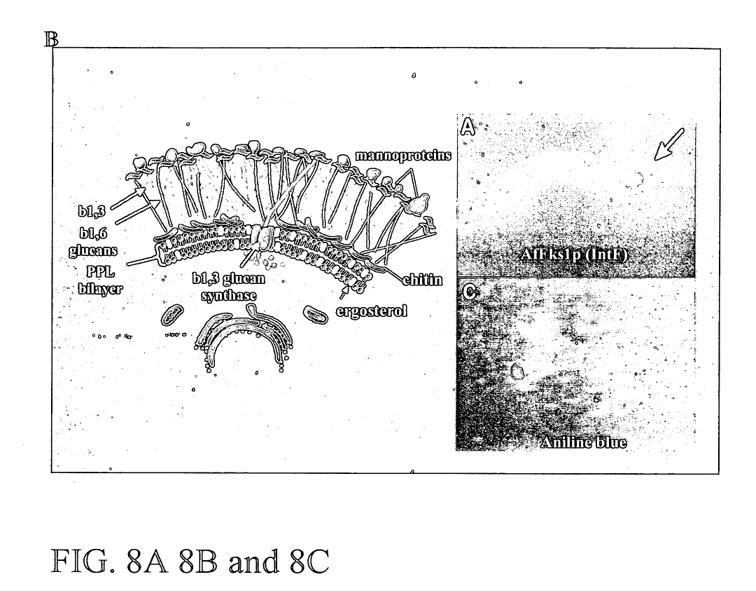

CAS is a novel antifungal agent that irreversibly inhibits the enzyme 1,3-β-D-glucan synthase, preventing the formation of glucan polymers and disrupting the integrity of the fungal cell wall (Bowman et al., 2002, FIG. 8A). This glucan synthase complex is located in the apical tips of Aspergillus hyphae (Beauvais et al., 2001, FIG. 8B, right panel).

Pharmacokinetic studies in humans have shown that the typical CAS plasma concentrations that are achieved in humans after the conventional intravenous CAS dosing (50-70 mg/day) exceed 1 μg/ml. For that reason, AF293 (Aspergillus) will be plated on YAG plates containing 2 μg/ml of CAS and conidia will be collected after 3 days. Conidia (suspensions of 10⁴ conidia/ml) will then be incubated at 37° C. for 16-20 h in liquid YAG medium containing 2 μg/ml of CAS to allow for germination to hyphae. Subsequently, CAS-exposed conidia or hyphae will be co-incubated with 10⁹ TU units of CX₇C phage display library. The steps outlined in FIG. 10 will be again followed. After 5 rounds of selection for CAS-exposed conidia and hyphae, the peptide-inserts of random phage clones are sequenced and the peptide sequences according to enrichment and by ClustalW sequence alignment are analyzed. Selected motifs may then again be used to search non-redundant protein databanks and validation of the specificity of the binding of the phages of interest will be done accordingly.

### Example 3

### In vivo Phage Display for Identification of Peptides that Home in Mouse Lung Tissues in the Setting of Acute Invasive Pulmonary Aspergillosis

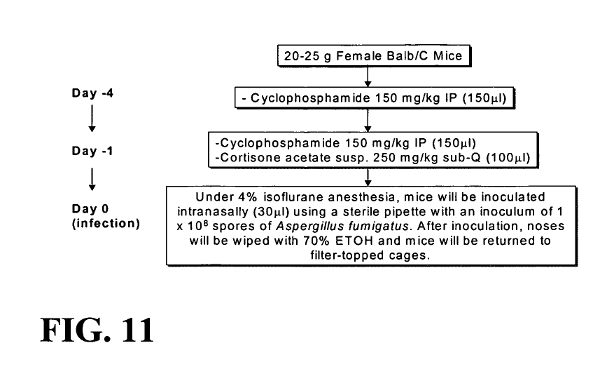

Murine model of acute IA: During the last 3 years a murine model of acute invasive pulmonary aspergillosis that mimics the pathogenesis of the infection has been established and it has been extensively used to test the efficacy of several antifungal agents against Aspergillus species (Lewis et al., 2002, Liu et al., 2003, Lewis and Kontoyiannis, 2001). FIG. 11 outlines the procedures used for immunosuppression and infection of the mice (via inhalation) with Aspergillus conidia.

After inoculation with Aspergillus conidia mice develop signs and symptoms of pneumonia (approximately 48-72 h after inoculation) that progresses to respiratory failure around 96-120 h after inoculation. The moribund animals are best identified by the presence of any of the following 4 criteria: 1. rapid breathing rate accompanied by intermittent slow, labored breathing, 2. ruffled fur, 3. hunched posture, and 4. hypothermia (animal cool to touch).

In vivo phage display: Aspergillus is an angiotropic mold that invades vessels and results in tissue infarcts. Identifying potential ligands that home to mouse lung tissue in the presence of acute IA could result in significant insights on potential ligands that might be expressed on the surface of human lung vasculature in the setting of acute invasive disease. In vivo selection of phage homing to the lung vasculature of mice with acute IA is performed. First the CX₇C-library in Balb/c mice without previous exposure to A. fumigatus 293 conidia is cleared and the unbound phage to isolate phage binding to the lung vasculature of mice with acute IA is used. Recovered phage will then be amplified and used for two subsequent rounds of selection. 30-100 phage clones are sequenced after 3 rounds of selection and the peptide sequences are analyzed according to enrichment and by ClustalW sequence alignment. Phage containing enriched peptide motifs will then be tested individually for their homing ability to the lung vasculature of Balb/c mice exposed to A. fumigatus compared to mice without previous exposure to that fungus. FIG. 5 summarizes the procedures of the in vivo selection (Pasqualini et al., 2000).

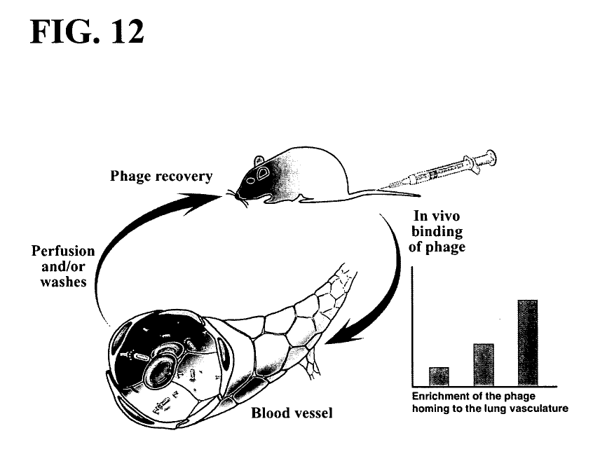

Identification of receptors for A. fumigatus-exposed lung vasculature homing ligands by using biochemical methods. See FIG. 12. The first approach to be used is affinity chromatography. If cell lines expressing a particular receptor can be found (this can be evaluated by using phage-binding assays) it will be possible to use such positive cell lines as the source of protein. Otherwise, receptors will be purified from a lung homogenate from mice with IA. Briefly, 300 000 cells will be incubated with 10⁹ TU of phage (peptide or antibody phage) of interest, and the cell/phage suspension will be spin through an organic phase in the BRASIL method (Giordano et al., 2001). Cell-bound phage will be isolated by bacterial infection and serial bacterial dilutions will be plated on LB/carbenicillin-plates to assess phage binding. A plasma membrane preparation (Spector et al., 1998) of phage binding cells will be used as the starting material for affinity chromatography. Commercially obtained synthetic peptides will be coupled to CnBr-activated sepharose. Plasma membrane preparations will be purified using the affinity columns in the presence of detergent (octyl-β-glucopyranoside) to isolate the proteins binding to selected peptides. The affinity-resin bound proteins will be eluted with the corresponding synthetic peptide and run in one-dimensional or two-dimensional SDS-PAGE gels. Once a protein band/spot in the material that binds to the peptide column and elutes specifically with the corresponding synthetic peptide is identified (from the SDS-PAGE gel), the options are to micro-sequence the protein or to prepare antibodies against it. If the sequence is new, it will be used to design oligonucleotide probes for the isolation of cDNA clones. Antibodies against the gel-isolated protein may also be used for the isolation of clones from bacterial expression libraries.

### Example 4

### CWGHSRDEC (SEQ ID NO:1) Peptide as Prophylaxis Against a Lethal Challenge of Acute Invasive Pulmonary Aspergillosis

Murine model of acute IA: During the last 3 years a murine model of acute invasive pulmonary aspergillosis has been established that mimics the pathogenesis of the infection and it has been used to test the efficacy of several antifungal agents against Aspergillus species (Lewis et al., 2002, Liu et al., 2003). After inoculation with Aspergillus conidia mice develop signs and symptoms of pneumonia (48-72 h after inoculation) that progresses to respiratory failure around 96-120 hours after inoculation. Hence, the model results in approximately 100% mortality by day +4 post-infection.

CWGHSRDEC (SEQ ID NO:1) synthetic peptide used as prophylaxis against IA: Mice are exposed to the CWGHSRDEC (SEQ ID NO:1) synthetic peptide before infecting them with A. fumigatus conidia and examined for any difference in mortality between mice pre-exposed to the CWGHSRDEC (SEQ ID NO: 1) peptide vs. mice not pre-exposed to the CWGHSRDE (SEQ ID NO: 1) peptide (which are expected to have 100% mortality by day +4). Since the CWGHSRDEC (SEQ ID NO:1) peptide is a ligand on the surface of Aspergillus conidia, the presence of it in the airways of mice could lead to the attachment of A. fumigatus conidia to the CWGHSRDEC (SEQ ID NO: 1) synthetic peptides and could thus prevent the attachment of A. fumigatus conidia to the tissue receptors that would normally permit invasion by A. fumigatus hyphae.

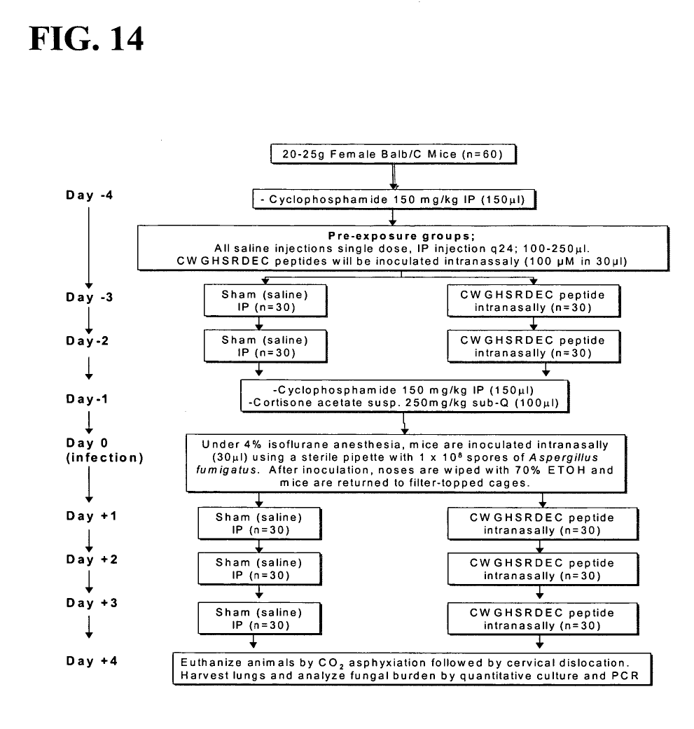

Four endpoints evaluated: Survival, b) Microscopic assessment of fungal burden in the lungs, c) Quantitative culture in the lungs and d) Quantitative PCR analysis from homogenized lung tissue (a very sensitive assay that correlates well with mortality, Bowman et al., 2001). FIG. 14 outlines the entire procedure.

Test the “cross-binding” of the two most frequently recovered phages from A. fumigatus conidia and hyphae. In order to assess whether the CWGHSRDEC (SEQ ID NO:1) and CGGRLGPFC (SEQ ID NO:8) phages are specific for AF293 conidia and hyphae respectively, the BRASIL method is used on the CWGHSRDEC (SEQ ID NO: 1) phage for AF293 hyphae and the CGGRLGPFC (SEQ ID NO:8) phage for AF293 conidia.

GGRLGPF (SEQ ID NO:]) phage binding to AF293 conidia. 1×10⁹ TU of the CGGRLGPFC (SEQ ID NO:8) phage is incubated with 200 μl of a 5×10⁸ AF293 conidia/ml solution in 1% BSA/PBS for 2 h at room temperature. Conidia/phage suspension is centrifuged over an organic phase for 10 min at 10,000 g and the pelleted conidia/phage is used for k91kan infection. Then the BRASIL method is followed as mentioned previously.

WGHSRDE(SEQ ID NO:1) phage binding to AF293 hyphae. 1×10⁹ TU of the CWGHSRDEC (SEQ ID NO:1) phage is incubated with 200 μl of a concentrated AF293 hyphal solution in 1% BSA/PBS for 2 h at room temperature. Hyphae/phage suspension is centrifuged over an organic phase for 20 min at 100000 g and the pelleted hyphae/phage is used for k91kan infection. Then the BRASIL method is followed as mentioned previously.

Binding of collagen VI to A. fumigatus conidia and microscopic examination of attachment. Sixteen microtiter wells of a 96-well microdilution plate are coated with 50 μl of 5 μg/ml of collagen VI. Another 16 wells are coated with 50 μl of a 3% BSA/PBS solution. The microdilution plate is left overnight at +4° C. and, the following day, the 32 wells are rinsed with PBS in order to remove any unbound collagen VI (or BSA). The wells are blocked with 200 μl of a 3% BSA/PBS solution for 2 hours at room temperature and rinsed again with PBS. After that, 50 μl of a 5×10⁸ conidia/ml AF293 solution (diluted in 1% BSA/PBS) is added in the 32 wells and incubated for 4 hrs at room temperature. The wells are washed gently with PBS and the amount of AF293 conidia attached to either collagen VI or BSA (negative control) is counted in multiple fields under a microscope.

Specific inhibition of the CWGHSRDEC (SEQ ID NO:1) phage binding on A. fumigatus conidia by the CWGHSRDEC (SEQ ID NO:1) peptide. To validate that the CWGHSRDEC (SEQ ID NO:1) phage is mediating specific binding to AF293 conidia, the CWGHSRDEC (SEQ ID NO:) phage and the AF293 conidia are co-incubated in the presence of increasing concentrations of the CWGHSRDEC (SEQ ID NO: 1) peptide. A decrease in the amount of CWGHSRDEC (SEQ ID NO: 1) phage binding on the surface of AF293 conidia after adding the CWGHSRDEC (SEQ ID NO:1) peptide indicates the CWGHSRDEC (SEQ ID NO:1) peptide binds to the surface of AF293 conidia thus preventing the corresponding phage from binding on these sites.

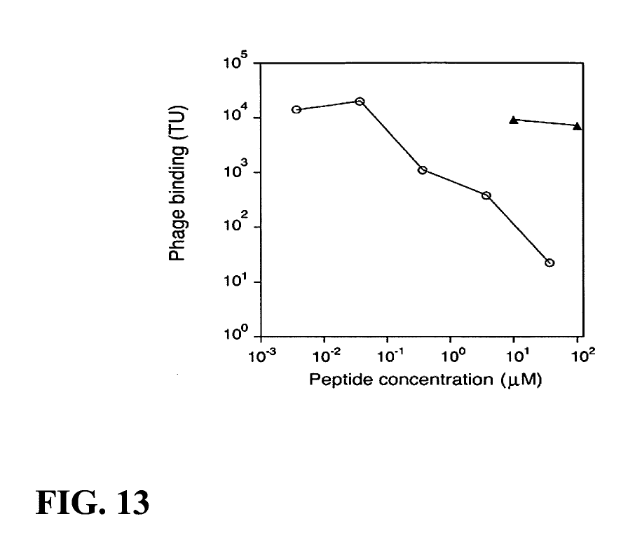

Protocol: Increasing concentrations of the synthetic CWGHSRDEC (SEQ ID NO:1) peptide or a control scramble peptide (e.g., CLLSATPSC (SEQ ID NO:)) (0.1, 1, 10 and 100 μM) is incubated with 200 μl of a 5×10⁸ AF293 conidia/ml solution in 1% BSA/PBS for 1 hour at room temperature and 10⁸ TU of CWGHSRDEC (SEQ ID NO:1) phage is added and incubated for 2 hours at room temperature. The conidia/phage suspension is centrifuged over an organic phase for 10 min at 10,000 g. Pelleted conidia/phage is used for k91kan infection. The BRASIL method is followed as previously mentioned. A potential decrease in the phage binding on AF293 conidia as a function of the addition of the CWGHSRDEC (SEQ ID NO:1) peptide is graphed as shown in FIG. 13 (Giordano et al, 2001):

Specific inhibition of the CWGHSRDEC (SEQ ID NO: 1) phage binding on A. fumigatus conidia by collagen VI. Increasing concentrations of the collagen VI (RDI, Research Diagnostics Inc.) (50 μl of 1, 2, 5 and 10 μg/ml collagen VI) is incubated with 200 μl of a 5×10⁸ AF293 conidia/ml solution in 1% BSA/PBS for 1 hour at room temperature and 10⁸ TU of CWGHSRDEC (SEQ ID NO: 1) phage is be added and incubated for 2 hours at room temperature. The conidia/phage suspension is centrifuged over an organic phase for 10 minutes at 10,000×g and the pelleted conidia/phage is used for infection of k91kan. The BRASIL method is followed as previously mentioned. A potential decrease in the CWGHSRDEC (SEQ ID NO: 1) phage binding to AF293 conidia as a function of the addition of increasing concentrations of collagen VI indicates that collagen VI mediates specific binding of A. fumigatus conidia.

Aspergillus conidial protein surface extraction. Chromatography using CWGHSRDEC (SEQ ID NO:1) peptide resin-based separation of surface proteins that bind to the CWGHSRDEC (SEQ ID NO:1) peptide. The CWGHSRDEC (SEQ ID NO:1) peptide and a control peptide is coupled either from the amino terminus to CNBr activated sepharose or from the carboxyl terminus to EAH activated sepharose at a concentration of 1-2 mg/ml. Cell surface proteins are extracted from AF293 conidia as described previously (Latge et al., 1999) and are run through an affinity chromatography column in PBS (and a suitable detergent depending on the protein extraction protocol). After an extensive washes of the column, bound proteins is eluted either with the CWGHSRDEC (SEQ ID NO: 1) peptide or with a low pH-buffer. Eluted proteins are separated by 1-dimensional and/or 2-dimensional gel electrophoresis. Proteins unique to CWGHSRDEC-peptide (SEQ ID NO: 1) will be sent for micro-sequencing.

In vitro validation that the CGGRLGPFC (SEQ ID NO:8) phage (the most commonly recovered phage bound on the surface of A. fumigatus hyphae) is specific for A. fumigatus hyphae—Specific inhibition of the CGGRLGPFC (SEQ ID NO:8) phage binding on A. fumigatus hyphae by the GGRLGPF (SEQ ID NO:9) peptide

Similarly as described earlier for the binding inhibition experiments with AF293 conidia and the CWGHSRDEC (SEQ ID NO: 1) phage and peptide, increasing concentrations of the synthetic CGGRLGPFC peptide (SEQ ID NO:8) or a control scramble peptide (e.g., CLLSATPSC (SEQ ID NO:) (0.1, 1, 10 and 100 μM) is incubated with 200 ti of a concentrated AF293 hyphal solution in 1% BSA/PBS for 1 hour at room temperature and 10⁸ TU of CGGRLGPFC (SEQ ID NO:8) phage is added and incubated for 2 hours at room temperature. The hyphae/phage suspension is centrifuged over an organic phase for 20 minutes at 100,000 g and pelleted conidia/phage is used for infection of k91kan. The BRASIL method is followed as previously mentioned. A potential decrease in the CGGRLGPFC (SEQ ID NO:8) phage binding to AF293 hyphae as a function of the addition of increasing concentrations of the CGGRLGPFC (SEQ ID NO:8) synthetic peptide indicates that the CGGRLGPFC (SEQ ID NO:8) peptide binds to the surface of AF293 hyphae thus preventing the corresponding phage from binding to these sites.

Sequencing and alignments. After each round the peptide inserts of 94 randomly selected phage clones were sequenced by DNA sequencing using the primer 5′-CCCTCATAGTTAGCGTAACGATCT-3′ (SEQ ID NO:17) and the Big Dye Terminator Cycle Sequencing Kit (Perkin Elmer, Norwalk, Conn.). Peptide sequences were aligned using the ClustalW alignment program (European Bioinformatics Institute homepage, (www2.ebi.ac.uk/clustalw/). Enriched peptide sequences were aligned to protein databases using the BLAST program of the National Center for Biotechnology Information www.ncbi.nlm.nih.gov/BLAST/). Similarity was defined as percentage of positive matches in the area aligned by the program.

Phage attachment and competition experiments. Binding of selected phage was examined with human adherent primary urothelial cells, the breast cancer cell line MDA-MB-435 and the transitional carcinoma cell lines RT4 and T24. All cells were grown to subconfluency in 48 well plates and free binding sites were blocked with 800 μl 30% FCS/DMEM (blocking medium) for 1 hour at 37° C. The blocking solution was then replaced by 200 μl 10% FCS/DMEM (washing medium) containing 1×108 cfu of each phage per well. After incubation for 2 hours at 4° C. to prevent unspecific endocytosis, unbound phage were removed by washing 7 times with 500 μl washing medium. For competition experiments increasing concentrations of the corresponding peptide or a control peptide (CARAC, SEQ ID NO:5) were added during the incubation. Bound phage were determined by infection with 500 μl log phase K91 culture and plating of serial dilutions. Values represent means of serial dilutions of triplicates wells and are given relative to binding of insertless fd-tet phage.

To determine binding to intact mucosa a novel dot blot chamber assay was developed, placing the bladder or ureter specimen into a dot blot chamber (Biorad, Hercules, Calif.), with the mucosa facing upwards, thus generating up to 96 equally large fields of mucosa. Blocking and washing in the dot blot chamber was performed as above but with 400 μl of the corresponding medium and infection was performed with 400 μl of log-phase K91kan culture per well. Three wells were pooled as one well.

Statistical Analysis of the Peptide Motifs. A system has been designed to analyze the data resulting from peptide library screenings, adapted from the SAS package. The system is available upon request from the M. D. Anderson Cancer Center.

All of the compositions, methods and apparatus disclosed and claimed herein can be made and executed without undue experimentation in light of the present disclosure. While the compositions and methods of this invention have been described in terms of preferred embodiments, it are apparent to those of skill in the art that variations maybe applied to the compositions, methods and apparatus and in the steps or in the sequence of steps of the methods described herein without departing from the concept, spirit and scope of the invention. More specifically, it are apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,472,509
U.S. Pat. No. 5,021,236
U.S. Pat. No. 5,206,347
U.S. Pat. No. 5,223,409
U.S. Pat. No. 5,401,511
U.S. Pat. No. 5,492,807
U.S. Pat. No. 5,603,872
U.S. Pat. No. 5,622,699
U.S. Pat. No. 5,670,312
U.S. Pat. No. 5,705,610
U.S. Pat. No. 5,840,841
U.S. Pat. No. 5,889,155
U.S. Pat. No. 6,068,829
Abruzzo et al., Antimicrob. Agents Chemother., 41(11):2333-2338, 1997.
Arap et al. Curr. Opin. Oncol., 1998b.
Arap et al., Nature Med. 8, 121-127, 2002.
Arap et al., Science, 279:377-380, 1998a.
Balow et al., J. Immunol., 114(3):1072-1076, 1975.
Barany and Merrifield, In: The Peptides, Gross and Meienhofer (Eds.), Academic Press, NY, 1-284, 1979.
Beauvais et al., J. Bacteriol., 183(7):2273-2279, 2001.
Bowman et al., Antimicrob. Agents Chemother., 46(9):3001-3012, 2002.
Brooks et al., Cell, 79, 1157-1164, 1994b
Burg et al., Cancer Res., 58:2869-2874, 1999.
Chaveroche et al., Nucleic Acids Res., 28(22):E97, 2000.
Clemons et al., Clin. Exp. Immunol., 122(2):186-191, 2000.
De Rosa et al., J. Pharm Sci., 91(3):790-799, 2002.
Ellerby et al., Nature Med., 9:1032-1038, 1999.
Espinel-Ingroff, J. Clin. Microbiol., 36(10):2950-2956 1998.
Folkman, Nature Biotechnol., 15: 510, 1997.
Folkman, Nature Med., 1:27-31, 1995.
Giordano et al., Nat. Med., 7:1249-1253, 2001.
Harlow and Lane, Antibodies: A Laboratory Manual (Cold Spring Harbor Laboratory Press, New York, N.Y.), 1988.
Johnson et al., In: Biotechnology And Pharmacy, Pezzuto et al. (Eds.), Chapman and Hall, NY, 1993.
Koivunen et al., J. Nucl. Med., 40:40:883-888, 1999.
Koivunen et al., Methods Mol. Biol., 129, 3-17, 1999b.
Koivunen et al., Nature Biotechnol., 17:768-774, 1999a
Kontoyiannis and Bodey, Eur. J. Clin. Microbiol. Infect. Dis., 21(3):161-172, 2002.
Latge, Clin. Microbiol. Rev., 12(2):310-350, 1999.
Lewis and Kontoyiannis, Pharmacotherapy, 21(8 Pt 2):149S-164S, 2001.
Lewis et al., Antimicrob. Agents Chemother., 46(11):3499-3505, 2002.
Liu et al., Antimicrob. Agents Chemother., 47(11):3592-3597, 2003.
Manual of Clinical Microbiology by Patrick R. Murray et al., American Society for Microbiology, 1999.
Merrifield, Science, 232: 341-347, 1986
Pasqualini et al., Cancer Res. 60: 722-727, 2000.
Pasqualini et al., In: Phage Display: A Laboratory Manual Barbas et al., (Eds.), 221:22-24, Cold Spring Harbor Laboratory Press, NY, 2000.
Pasqualini nd Ruoslahti, Nature, 380:364-366, 1996.
Pasqualini, Quart. J. Nucl. Med., 43:159-162, 1999.
PCT Appln. PCT/US01/28124
Pfaller, J. Hosp. Infect., 30:329-38, 1995, 1995.
Rajotte and Ruoslahti, J. Biol. Chem., 274:11593-11598, 1999.
Rajotte et al., J. Clin. Invest., 102:430-437, 1998
Remington's Pharmaceutical Sciences, 15th ed., pp. 1035-1038 and 1570-1580, 1990.
Smith and Scott, Meth. Enzymol., 21:228-257, 1993.
Smith and Scott, Science, 228:1315-1317, 1985
Spector et al., J. Clin. Invest., 101(2):497-502, 1998.
Stewart and Young, Solid Phase Peptide Synthesis, 2d. ed., Pierce Chemical Co., 1984.
Tam et al., J. Am. Chem. Soc., 105:6442, 1983. De Rosa et al., Int. J. Pharm., 242 (1-2):225, 2002.
The Use of Antibiotics: A Clinical Review of Antibacterial, Antifungal and Antiviral Drugs, Kucers et al., Butterworth-Heinemann, 1997.

1. A composition comprising an isolated peptide having at least 3 contiguous amino acids of a sequence selected from the group consisting of SEQ ID NO: 1 through SEQ ID NO:16, wherein said peptide binds to fungal cells.

2. The composition of claim 1, wherein the peptide is 50 amino acids or less in size.

3. The composition of claim 1, wherein the peptide is 25 amino acids or less in size.

4. The composition of claim 1, wherein the peptide is 10 amino acids or less in size.

5. The composition of claim 1, wherein the peptide is 9 amino acids or less in size.

6. The composition of claim 1, wherein the peptide is 7 amino acids or less in size.

7. The composition of claim 1, wherein the peptide comprises at least 5 contiguous amino acids of a sequence selected from the group consisting of SEQ ID NO: 1 through SEQ ID NO:16.

8. The composition of claim 7, wherein the peptide is operatively coupled to an agent to be delivered to a fungal cell.

9. The composition of claim 8, wherein the peptide is covalent coupled to the agent.

10. The composition of claim 8, wherein the agent is a drug, a chemotherapeutic agent, a radioisotope, an anti-fungal agent, a peptide, a protein, an antibiotic, an antibody, a Fab fragment of an antibody, an imaging agent, a cell, a vector or a virus.

11. The composition of claim 8, wherein the fungal cell is an Aspergillus, Fusarium, Zygomycetes or Scedosporium cell.

12. The composition of claim 10, wherein the anti-fungal agent comprises one or more agents selected from the group consisting of voriconazole, posaconazole, clotrimazole, miconazole, ketoconazole, econazole, butoconazole, oxiconazole, terconazole, itraconazole, ravuconazole, fluconazole, amphotericin B, amphotericin B lipid formulation, liposomal amphotericin B, ABLC, nystatin, nystatin lipid formulation, an azole, terbinafine, echinocandin, terbinafine, naftifine, tolnaftate, mediocidin, candicidin, pimaricin, trichomycin, hamycin, aurefungin, ascosin, ayfattin, azacolutin, trichomycin, levorin, heptamycin, candimycin, perimycin, caspofungin, micafungin, anidulfungin.

13. The composition of claim 8, wherein the agent is a liposome.

14. The composition of claim 1, wherein the peptide is attached to a solid support.

15. The composition of claim 1, wherein said peptide selectively binds to fungal cells.

16. The composition of claim 1, wherein the peptide is at most 100 residues.

17. The composition of claim 16, wherein the peptide is at most 50 residues.

18. The composition of claim 17, wherein the peptide is 20 to 50 residues in length.

19. The composition of claim 16, wherein the peptide is 7 to 13 residues in length.

20. The composition of claim 1, wherein the peptide is a cyclic peptide.

21. The composition of claim 1, further comprising lipids.

22. The composition of claim 21, wherein the lipids include phospholipids in the form of liposomes.

23. A method of selecting a fungal cell targeting peptide comprising:
a) obtaining at least one sample comprising fungal cells;
b) exposing the sample to a peptide library; and
c) recovering one or more peptides that bind to the fungal cells.

24. The method of claim 23, wherein the peptide library is a phage display library.

25. The method of claim 24, wherein phage are recovered by infecting pilus positive bacteria.

26. The method of claim 24, wherein said phage are recovered by:
a) amplifying phage inserts;
b) ligating the amplified inserts to phage DNA; and
c) producing phage from the ligated DNA.

27. The method of claim 24, wherein phage are recovered by BRASIL (Biopanning and Rapid Analysis of Selective Interactive Ligands).

28. The method of claim 23 further comprising obtaining one or more types of non-fungal cells and exposing said cells to said peptide library and recovering one or more peptides that do not bind to said one or more types of non-fungal cells.

29. The method of claim 28, further comprising:
a) preselecting the phage library against non-fungal cell type;
b) removing phage that bind to the non-fungal cell type; and
c) selecting the remaining phage against fungal cells.

30. The method of claim 23, wherein the fungal cells are Aspergillus, Fusarium, Zygomycetes or Scedosporium cells.

31. A method of treating a fungal infection in a subject comprising:
a) obtaining a fungal cell targeting peptide wherein the peptide i) is operatively coupled to and delivers a therapeutic agent to the fungal cell, ii) inhibits the adhesion of the fungal cell to a tissue or organ, or iii) is operatively coupled to and delivers a therapeutic agent to the fungal cell and inhibits the adhesion of the fungal cell to a tissue or organ; and
b) administering the peptide to the subject.

32. The method of claim 31, wherein the targeting peptide comprises a peptide having at least 3 contiguous amino acids of a sequence selected from the group consisting of SEQ ID NO: 1 through SEQ ID NO:16, wherein said peptide binds to fungal cells.

33. The method of claim 32, wherein the targeting peptide comprises a peptide having at least 5 contiguous amino acids of a sequence selected from the group consisting of SEQ ID NO: 1 through SEQ ID NO:16, wherein said peptide binds to fungal cells.

34. The method of claim 31, wherein the therapeutic agent is a drug, a chemotherapeutic agent, a radioisotope, an anti-fungal agent, a peptide, a protein, an antibiotic, an antibody, a Fab fragment of an antibody, an imaging agent, a cell, a vector or a virus.

35. The method of claim 34, wherein the anti-fungal agent comprises one or more agents selected from the group consisting of voriconazole, posaconazole, clotrimazole, miconazole, ketoconazole, econazole, butoconazole, oxiconazole, terconazole, itraconazole, ravuconazole, fluconazole, amphotericin B, amphotericin B lipid formulation, liposomal amphotericin B, ABLC, nystatin, nystatin lipid formulation, an azole, terbinafine, echinocandin, terbinafine, naftifine, tolnaftate, mediocidin, candicidin, pimaricin, trichomycin, hamycin, aurefungin, ascosin, ayfattin, azacolutin, trichomycin, levorin, heptamycin, candimycin, perimycin, caspofungin, micafungin, anidulfungin.

36. A method of targeting the delivery of an agent to a fungal cell in a subject, comprising:
a) obtaining a fungal cell targeting peptide composition;
b) operatively coupling the peptide to the agent; and
c) administering the peptide-coupled agent to the subject.

37. The method of claim 36, wherein the subject is a human, a mouse, a dog, a cat, a rat, a sheep, a horse, a cow, a goat or a pig.

38. The method of claim 36, wherein the agent is a drug, a chemotherapeutic agent, a radioisotope, an anti-fungal agent, a peptide, a protein, an antibiotic, an antibody, a Fab fragment of an antibody, an antigen, an imaging agent, a cell, a vector or a virus.

39. A method of identifying a fungal cell, comprising:
a) contacting a sample suspected of comprising a fungal cell with an isolated fungal cell targeting peptide; and
b) detecting binding of the peptide to the sample, thereby identifying sample as comprising fungal cells.

40. A method of identifying a receptor or protein that interacts with a fungal targeting peptide, comprising the steps of:
a) obtaining a composition suspected of comprising a receptor or protein that interacts with a fungal cell targeting peptide;
b) contacting the composition with a fungal cell targeting peptide under conditions that permit binding of the peptide to any such receptor or protein present in the composition; and
c) identifying a receptor or protein that binds to the peptide.

41. The method of claim 40, wherein the composition comprises fungal cells.

42. The method of claim 40, further comprising isolating the receptor or protein.

43. The method of claim 40, further comprising preparing an antibody or antibody fragment that recognizes and binds to the receptor or protein.

44. The method of claim 43, further comprising attaching an agent that one desires to have delivered to fungal cells to said antibody or antibody fragment.

45. An antibody or antibody fragment that recognizes and binds to a receptor or protein identified by the method of claim 40.

46. The antibody or antibody fragment of claim 45, further comprising an agent or macromolecular complex that one desires to have delivered to fungal cells attached to said antibody or antibody fragment.

47. A method of selectively targeting a fungal cell in a patient, comprising the steps of:
a) obtaining an antibody or antibody fragment in accordance with claim 45 or prepared by the method of claim 44; and
b) administering the antibody or fragment to said patient to thereby target the fungal cells.


SEQUENCE LISTING (17 sequences)

SEQ ID NO: 1 - protein, 7 aa, Aspergillus fumigatus
WGHSRDE

SEQ ID NO: 2 - protein, 7 aa, Aspergillus fumigatus
LLSATPS

SEQ ID NO: 3 - protein, 7 aa, Aspergillus fumigatus
GARASGS

SEQ ID NO: 4 - protein, 7 aa, Aspergillus fumigatus
ELRRGGS

SEQ ID NO: 5 - protein, 7 aa, Aspergillus fumigatus
SSGDRTA

SEQ ID NO: 6 - protein, 7 aa, Aspergillus fumigatus
SLVRGGT

SEQ ID NO: 7 - protein, 7 aa, Aspergillus fumigatus
IGRAPQM

SEQ ID NO: 8 - protein, 7 aa, Aspergillus fumigatus
GGWGPGS

SEQ ID NO: 9 - protein, 7 aa, Aspergillus fumigatus
GGRLGPF

SEQ ID NO: 10 - protein, 7 aa, Aspergillus fumigatus
DLTHVSA

SEQ ID NO: 11 - protein, 7 aa, Aspergillus fumigatus
GTSRWLR

SEQ ID NO: 12 - protein, 7 aa, Aspergillus fumigatus
  MOD_RES (1): Xaa = can be any naturally occuring amino acid
XCDSSVS

SEQ ID NO: 13 - protein, 7 aa, Aspergillus fumigatus
VVGSADG

SEQ ID NO: 14 - protein, 7 aa, Aspergillus fumigatus
ISTFARG

SEQ ID NO: 15 - protein, 7 aa, Aspergillus fumigatus
VGVEYRT

SEQ ID NO: 16 - protein, 7 aa, Aspergillus fumigatus
NPGYWGN

SEQ ID NO: 17 - DNA, 24 bp, Artificial Sequence
  Description of Artificial Sequence  Synthetic Primer
ccctcatagttagcgtaacgatct